(19) United States
(12) Patent Application Publication
(10) Pub. No.: US 2025/0213355 A1
(43) Pub. Date: Jul. 3, 2025

(54) METHODS AND SYSTEMS FOR MATERIAL MODIFICATIONS USING LASER TECHNIQUES

(71) Applicant: EDWARDS LIFESCIENCES CORPORATION, Irvine, CA (US)

(72) Inventors: Ekaterina Tkatchouk, Huntington Beach, CA (US); Carolyn Sue Martinez, Petaluma, CA (US); Austin Engelbrecht, Trabuco Canyon, CA (US); Nazanin Afsar Kazerooni, Costa Mesa, CA (US)

(21) Appl. No.: 19/061,607

(22) Filed: Feb. 24, 2025

Related U.S. Application Data
(63) Continuation of application No. PCT/US2023/030296, filed on Aug. 15, 2023.
(60) Provisional application No. 63/373,515, filed on Aug. 25, 2022.

(51) Int. Cl. A61F 2/24

(52) U.S. Cl. CPC A61F 2/2415; A61F 2/2418; A61F 2220/0075

(57) ABSTRACT

Systems and methods for improving biocompatibility and mechanical properties of cardiac repair and regenerative devices are described. Laser techniques can be applied for material modification of various parts of cardiac repair and regenerative devices including patterning on the frame, leaflets, and/or skirt material. Material modification can aid in healing, tissue acceptance, and/or anchoring of the prosthetic valve.

### CROSS-REFERENCE TO RELATED APPLICATIONS

This application is a continuation of International Application No. PCT/US2023/030296, filed Aug. 15, 2023, which claims the benefit of U.S. Patent Application No. 63/373,515, filed Aug. 25, 2022, the entire disclosures all of which are incorporated by reference for all purposes.

### FIELD OF THE DISCLOSURE

The present disclosure relates to methods and systems to produce materials, biomaterials, components, devices, implants, and systems used in cardiac repair and regenerative devices. More specifically, systems and methods to modify cardiac repair devices via laser techniques to promote mechanical properties and/or biocompatibility such as endothelization into the materials and devices to allow tissue regeneration and incorporation of native tissue.

### BACKGROUND OF THE DISCLOSURE

Many of the current materials/components used in cardiovascular repair devices can have issues with biocompatibility. These materials can activate an immune response by forming an avascular fibrous capsule which can isolate and wall off an implantable device from the blood stream and can create issues with long term implant functionality.

One way of preventing encapsulation or an accompanying immune response is by promoting endothelial cell growth. The stimulation of endothelization on blood-contacting surfaces is thought to be a critical step in establishing the long-term biocompatibility of cardiovascular devices. Biological tissue, such as animal pericardium (e.g., bovine, porcine), currently used in medical devices and implantable bioprosthesis, such as bioprosthetic heart valves and vascular grafts is mostly composed of crosslinked collagen. One deficiency of cross-linked biological tissue used in bioprosthesis is that the act of crosslinking promotes calcification or tissue overgrowth and pannus formation. As such, there is a need for a novel synthetic or natural material with the ability to promote reendothelization.

### SUMMARY OF THE DISCLOSURE

Methods and systems for improving biocompatibility and/or mechanical properties of cardiac repair and regenerative devices are described.

In some implementations, a method to improve biocompatibility of a structure for use in a prosthetic comprises applying a laser process to at least one surface of the structure to create at least one pattern on the at least one surface; where the laser process comprises at least one laser source; the laser process is selected from the group consisting of direct laser writing, interference lithography, and any combinations thereof; and the structure is at least a component of a device selected from the group consisting of a prosthetic heart valve, a stent, and a cardiac patch.

In some implementations, the component is selected from the group consisting of a frame, a stent, a skirt, an outer skirt, an inner skirt, a suture, a leaflet, and a valve tissue.

In some implementations, the surface comprises a material selected from the group consisting of a metal, a metal alloy, a stainless steel, nitinol, titanium, Co—Cr alloy, a polymer, polymethylmethacrylate, polyetherketone, polyimide, polyamide, polyethylene, polytetrafluorethylene, nylon, polydimethylsiloxane, silicone, polyethylene terephthalate, polybutylene terephthalate, polyester, biopolymer, a blocked copolymer of polycarbonate, a poly(sulfone of bisphenol-A) (PSU)-PBT copolymer, collagen, acrylate collagen, chitosan, and a pericardial tissue.

In some implementations, the at least one laser source is an ultrashort pulse laser.

In some implementations, the ultrashort pulse laser has a pulse width from 3 picoseconds to 50 femtoseconds.

In some implementations, the at least one laser source has an emission wavelength selected from the group consisting of an infrared wavelength from 700 nm to 1 mm, a near infrared wavelength from 800 nm to 2500 nm, a visible light wavelength from 380 nm to 750 nm, and an ultraviolet wavelength from 100 nm to 400 nm.

In some implementations, the at least one laser source has an emission wavelength selected from the group consisting of 10.6 μm, 1060 nm, 1030 nm, 530 nm, 515 nm, 370 nm, 355 nm, 343 nm, 248 nm, and 193 nm.

In some implementations, the direct laser writing is carried out using a direct laser writing system comprising at least one laser beam, at least one substrate, and at least one galvaometric mirror.

In some implementations, the at least one substrate is fixed and the at least one galvaometric mirror moves the at least one laser beam to create a plurality of patterns; or the at least one laser beam is fixed and the at least one substrate moves to create the plurality of patterns; or the at least one substrate and the at least one laser beam move simultaneously to create the plurality of patterns.

In some implementations, the direct laser writing system comprises a focusing optic selected from the group consisting of a microscope objective, and an f-theta lens.

In some implementations, the at least one pattern improves reendothelization and tissue regeneration of the structure.

In some implementations, the laser process changes at least one property selected from the group consisting of surface topography, thickness, and dimension, of the structure.

In some implementations, the at least one surface is flat, curved, even, or uneven.

In some implementations, the at least one pattern is periodic or aperiodic.

In some implementations, the at least one pattern has at least one dimension in a range from 1 nm to 1 mm.

In some implementations, the laser process is a part of a subtractive process or an additive process.

In some implementations, the laser process is a laser ablation process, wherein the at least one pattern changes a thickness of the at least one surface.

In some implementations, the laser ablation process contours and creates different thickness on the at least one surface.

In some implementations, the at least one pattern comprises a hierarchical structure or is multi-dimensional.

In some implementations, the at least one pattern comprises a pattern selected from the group consisting of a line, a straight line, a curved line, a groove, a pillar, a pore, a ridge, a wave, a dimple, a square, and any combinations thereof.

In some implementations, the at least one pattern has at least one shape selected from the group consisting of circular, ovular, oblong, triangular, quadrilateral, rectangular, square, rhomboidal, trapezoidal, hexagonal, octagonal, and any combinations thereof.

In some implementations, the at least one pattern comprises parallel rows.

In some implementations, applying at least one chemical reagent to the at least one surface before applying the laser process, wherein the laser process generates a surface coating of the at least one surface.

In some implementations, the surface coating changes the contact angle of the at least one surface.

In some implementations, a method to modify a biomaterial for use within a prosthetic comprises adding a photoactive reagent to a proteinaceous material; and applying a laser process comprising at least one laser source onto the proteinaceous material; where the at least one laser source locally crosslinks the photoactive reagent with the proteinaceous material and changes a chemical structure of the proteinaceous material; and the proteinaceous material is at least a component of a system or device selected from the group consisting of a prosthetic heart valve, a stent, and a cardiac patch.

In some implementations, the photoactive reagent is selected from the group consisting of aryl azide, azido-methyl-coumarin, benzophenone, anthraquinone, diazo compound, diazirines psoralen derivative, vitamin B1 (thiamine), vitamin B2 (riboflavin), vitamin B3 (niacin), vitamin B5 (pantothenic acid), vitamin B6 (pyridoxine), vitamin B7 (biotin), vitamin B12 (cobalamin), folic acid, n-hydroxy succinimide ester of acrylic acid (ANHS), 2-hydroxy-4′-(2-hydroxyethoxy)-2-methylpropiophenone (Irgacure 2959 photoinitiator), methyl phenylglyoxylate, phenylbis(2,4,6-trimethylbenzoyl) phosphine oxide, sulfo-LC-SDA (Diazirine), triethylamine, deoxyribonucleic acid, bis-acylphosphine oxide, pyrolipid, porphyrin, and chlorin.

In some implementations, the photoactive reagent is added to the proteinaceous material by soaking the proteinaceous material in a solution comprising the photoactive reagent; or by surface coating the proteinaceous material with a solution comprising the photoactive reagent.

In some implementations, the photoactive reagent is riboflavin and the proteinaceous material comprises collagen.

In some implementations, the at least one laser source is an ultrashort pulse laser.

In some implementations, the at least one laser source has an emission wavelength from 100 nm to 400 nm.

In some implementations, the crosslink changes at least one mechanical property of the biomaterial selected from the group consisting of ultimate tensile strength, fatigue strength, and Young's modulus.

In some implementations, the laser process creates a gradient of crosslinking in the proteinaceous material by controlling the at least one laser source.

In some implementations, a prosthetic heart valve comprises an annular frame that is radially collapsible to a collapsed configuration and radially expandable to an expanded configuration. The frame has an inflow end and an outflow end, and defines a longitudinal axis along a lumen of the prosthetic heart valve when the prosthetic heart valve is in the expanded configuration. The prosthetic heart valve comprises a leaflet structure positioned within the frame and secured thereto. The prosthetic heart valve comprises a skirt comprising an inner skirt positioned on the inside of the frame and an outer skirt positioned on the outside of the frame. The inner skirt and the outer skirt are attached to at least a portion of the frame by a plurality of sutures. On the outside of the frame, the outer skirt extends along the longitudinal axis in an upstream direction and doubles back toward the outflow end of the frame at a fold line to form a cuff, and an edge portion of the outer skirt is secured to the outer skirt downstream of the fold line such that the cuff forms an inflow end of the laminate sealing member. For at least one surface of: the frame, the leaflet, the skirt, and the suture, is modified by a laser process comprising at least one laser source; and the laser process improves reendothelization and tissue regeneration of the prosthetic heart valve.

In some implementations, the laser process is selected from the group consisting of direct laser writing, interference lithography, and any combinations thereof.

In some implementations, the at least one laser source is an ultrashort pulse laser.

In some implementations, the ultrashort pulse laser has a pulse width from 1 millisecond to 1 femtosecond.

In some implementations, the at least one laser source has an emission wavelength selected from the group consisting of an infrared wavelength from 700 nm to 1 mm, a near infrared wavelength from 800 nm to 2500 nm, a visible light wavelength from 380 nm to 750 nm, and an ultraviolet wavelength from 100 nm to 400 nm.

In some implementations, the at least one laser source has an emission wavelength selected from the group consisting of 10.6 μm, 1060 nm, 1030 nm, 530 nm, 515 nm, 370 nm, 355 nm, 343 nm, 248 nm, and 193 nm.

In some implementations, the direct laser writing is carried out using a direct laser writing system comprising at least one laser beam, at least one substrate, and at least on galvaometric mirror.

In some implementations, the at least one substrate is fixed and the at least one galvaometric mirror moves the at least one laser beam to create a plurality of patterns; or the at least one laser beam is fixed and the at least one substrate moves to create the plurality of patterns; or the at least one substrate and the at least one laser beam move simultaneously to create the plurality of patterns.

In some implementations, the direct laser writing system comprises a focusing optic selected from the group consisting of a microscope objective, and an f-theta lens.

In some implementations, the at least one surface is flat, curved, even, or uneven.

In some implementations, the at least one surface comprises at least one pattern created by the laser process.

In some implementations, the at least one pattern is periodic or aperiodic.

In some implementations, the at least one pattern has at least one dimension in a range from 1 nm to 1 mm.

In some implementations, the at least one pattern comprises a hierarchical structure or is multi-dimensional.

In some implementations, at least one pattern is selected from the group consisting of a line, a straight line, a curved line, a groove, a pillar, a pore, a ridge, a wave, a dimple, a square, and any combinations thereof.

In some implementations, at least one pattern has at least one shape selected from the group consisting of circular, ovular, oblong, triangular, quadrilateral, rectangular, square, rhomboidal, trapezoidal, hexagonal, octagonal, and any combinations thereof.

In some implementations, the at least one pattern comprises parallel rows.

In some implementations, the laser process is a part of a subtractive process or an additive process.

In some implementations, the laser process is a laser ablation process, wherein the laser ablation process changes a thickness of the at least one surface.

In some implementations, the laser ablation process contours and creates different thickness on the at least one surface.

In some implementations, the laser process generates a surface coating on the at least one surface with at least one chemical reagent applied to the at least one surface.

In some implementations, the surface coating changes the contact angle of the at least one surface.

In some implementations, the leaflet comprises a pericardial tissue and the laser process generates a consistent thickness throughout the leaflet.

In some implementations, the laser process creates a pattern on the at least one surface of the frame and the pattern allows an easy tissue ingrowth and prevents a paravalvular leak.

In some implementations, the laser process modifies a surface energy and changes a contact angle of the at least one surface of the skirt or the suture.

In some implementations, a prosthetic heart valve comprises an annular frame that is radially collapsible to a collapsed configuration and radially expandable to an expanded configuration, The frame has an inflow end and an outflow end, and defines a longitudinal axis along a lumen of the prosthetic heart valve when the prosthetic heart valve is in the expanded configuration. The prosthetic heart valve comprises a leaflet structure positioned within the frame and secured thereto. The prosthetic heart valve comprises a skirt comprising an inner skirt positioned on the inside of the frame and an outer skirt positioned on the outside of the frame. The inner skirt and the outer skirt are attached to at least a portion of the frame by a plurality of sutures. On the outside of the frame, the outer skirt extends along the longitudinal axis in an upstream direction and doubles back toward the outflow end of the frame at a fold line to form a cuff, and an edge portion of the outer skirt is secured to the outer skirt downstream of the fold line such that the cuff forms an inflow end of the laminate sealing member. A laser process comprising at least one laser source locally crosslinks the leaflet with a photoactive reagent. The leaflet comprises a proteinaceous material, and the laser process changes a chemical structure of the proteinaceous material.

In some implementations, the photoactive reagent is selected from the group consisting of aryl azide, azido-methyl-coumarin, benzophenone, anthraquinone, diazo compound, diazirines psoralen derivative, vitamin B1 (thiamine), vitamin B2 (riboflavin), vitamin B3 (niacin), vitamin B5 (pantothenic acid), vitamin B6 (pyridoxine), vitamin B7 (biotin), vitamin B12 (cobalamin), folic acid, n-hydroxy succinimide ester of acrylic acid (ANHS), 2-hydroxy-4′-(2-hydroxyethoxy)-2-methylpropiophenone (Irgacure 2959 photoinitiator), methyl phenylglyoxylate, phenylbis(2,4,6-trimethylbenzoyl) phosphine oxide, sulfo-LC-SDA (Diazirine) and triethylamine, deoxyribonucleic acid, bis-acylphosphine oxide, pyrolipid, porphyrin, and chlorin.

In some implementations, the photoactive reagent is added to the proteinaceous material by soaking the proteinaceous material in a solution comprising the photoactive reagent; or by surface coating the proteinaceous material with a solution comprising the photoactive reagent.

In some implementations, the photoactive reagent is riboflavin and the proteinaceous material comprises collagen.

In some implementations, the at least one laser source is an ultrashort pulse laser.

In some implementations, the at least one laser source has an emission wavelength from 100 nm to 400 nm.

In some implementations, the crosslink changes at least one mechanical property of the material selected from the group consisting of ultimate tensile strength, fatigue strength, and Young's modulus.

In some implementations, the laser process creates a gradient of crosslinking in the proteinaceous material by controlling the at least one laser source.

Additional implementations and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIGS. 1A-1B illustrate exemplary modification processes using laser techniques.

FIGS. 2A-2C illustrate schematics of laser interference lithography system.

FIG. 3 illustrates a block diagram of direct laser writing.

FIGS. 4A-4B illustrate examples of various patterns generated via laser techniques.

FIGS. 5A-5B illustrate examples of various patterns generated via laser techniques on soft materials.

FIG. 6A-6B illustrate examples of hierarchical patterns generated using combinations of interference lithography and direct laser writing processes.

FIGS. 7A-7C illustrate examples of various patterns generated via laser techniques on hard materials.

FIGS. 8A-8B illustrate examples of laser ablation modification on biological materials.

FIG. 9 illustrates examples of laser assisted localized crosslinking of collagen with riboflavin.

FIG. 10 illustrates examples of laser modifications on a bioprosthetic heart valve.

### DETAILED DESCRIPTION OF THE DISCLOSURE

Turning now to the drawings, methods and systems for improving biocompatibility and/or mechanical properties of cardiac repair and regenerative devices are described. Many aspects provide modifications including (but not limited to) topography, surface patterning, mechanical property, chemistry, and surface chemistry, of materials including (but not limited to) soft materials, hard materials, biological materials, and synthetic materials. Examples of materials include (but are not limited to) stainless steels, metals, metal alloys, ceramics, polymers, textiles, biomaterials, biopolymers, pericardial tissues, and collagens. As can readily be appreciated, any of a variety of materials can be modified as appropriate to the requirements of specific applications in accordance with various examples of the invention. In several instances, the materials are part of cardiac repair and regenerative devices including (but not limited to) heart valves, prosthetic heart valves, transcatheter heart valves, mitral heart valves, aortic valves, prosthetic valves, stents, vascular stents, patches, vascular patches, and cardiac patches. As can readily be appreciated, any of a variety of biomedical devices can be modified as appropriate to the requirements of specific applications in accordance with various examples of the invention. In certain instances, the materials can be used to make components of a cardiac repair and regenerative device including (but not limited to) sutures, leaflets, prosthetic tissues, skirts, outer skirts, textile skirts, stents, and frames. In some instances, various micropatterns and/or nanopatterns can be generated on the surfaces of the materials. The surface patterns can be generated via laser techniques. Surface patterning shown in many examples can improve biocompatibility of the materials including (but not limited to) accelerating endothelization, improving cell adhesion, and modulating cell functions. In many instances, laser modification can create anti-thrombotic surface morphology. The laser modified surface can resist thrombus formation. In several examples, laser modification can provide more consistent thermal properties and/or mechanical properties throughout the materials. Examples of mechanical properties include (but are not limited to) fatigue resistance, tear propagation rates, ultimate tensile strength in uniaxial and multiaxial modes, and Young's modulus in dynamic and static modes. In some examples, laser modification can provide controlled and consistent thickness throughout the materials.

Many examples show that at least one surface of the materials of the cardiac repair and regenerative devices can be modified to have improved biocompatibilities including (but not limited to) endothelization and tissue regeneration. In several examples, the modification methods can be applied to materials that are suitable for the construction of devices and/or apparatuses that can be used in cardiac and/or vascular repair. Examples of such devices include (but are not limited to) prosthetic heart valves, stents, and cardiac patches. A number of examples show that the modification methods can be applied to materials that can be used for systems to deliver repair devices including (but not limited to) delivery devices and catheters.

Various methods are directed to improving biocompatibility and the potential of endothelization of materials by using laser techniques. In some instances, lasers can pattern and/or modify surfaces of the materials to mimic the chemistry and/or structure and morphology of naturally occurring extracellular matrices (ECM). Major components of ECM network can include collagen, proteoglycans and fibronectin, which can form part of three-dimensional structures with round cavities with different diameters ranging between about 10 microns and about 100 microns. Laser techniques can produce similar morphologies as ECM in various materials to allow cell to proliferate easily in accordance with several examples. In some instances, laser techniques can enable localized and/or gradient crosslink with collagen matrix to modify mechanical properties of the scaffold. In certain instances, laser techniques can improve surface chemistry and biocompatibility.

In several examples, laser techniques can modify mechanical properties of various materials. Mechanical properties including (but not limited to) tensile strength and fatigue resistance can be modified by adding chemical reagents including (but not limited to) photoactive or photo-reagent crosslinkers to various materials. UV light laser can be used to create reactive species and form new chemical bonding in the materials. Examples of the materials include (but are not limited to) soft materials, polymers, biomaterials, proteins, polysaccharides, collagens, chitosan, materials from biological sources, and pericardium tissues.

In certain examples, laser techniques can modify the thickness and/or structures of various materials. Thickness of a material can be modified by laser ablation, which may employ pulsed lasers to remove materials from a substrate for generating micro- and/or nano-structures. Many materials used in medical devices can be subjected to laser ablation. Laser ablation can create contouring on various materials including (but not limited to) medical grade polymers, medical grade metals and metal alloys, and materials from biological sources. Examples of medical grade polymers include (but are not limited to) polymethylmethacrylate, polyetherketone, polyimide, polyamide, polyethylene, polytetrafluorethylene, nylon, polydimethylsiloxane, silicone, polyethylene terephthalate, polyester (PET), polybutylene terephthalate (PBT), polyurethanes, blocked copolymer of polycarbonate, poly(sulfone of bisphenol-A) (PSU)-PBT copolymers. Examples of medical grade metals include (but are not limited to) nitinol and titanium. Examples of materials from biological sources include (but are not limited to) collagen, acrylate collagen composite, chitosan, and pericardium tissue. As can readily be appreciated, any of a variety of materials can be modified as appropriate to the requirements of specific applications in accordance with various examples of the invention. In many examples, pulsed lasers may provide a high efficiency in material removal. The pulsed lasers including (but not limited to) ultrashort pulsed (USP) lasers, may have pulse widths ranging from milli seconds to sub-pico seconds. Various light sources can be employed for ablation in medical devices. Examples of light sources include (but are not limited to) neodymium-doped yttrium aluminum garnet (Nd:YAG), excimer, carbon dioxide (CO₂), and fiber. As can readily be appreciated, any of a variety of laser light sources can be used as appropriate to the requirements of specific applications in accordance with various examples of the invention. Emission wavelengths can center in various ranges of the optical spectrum. In some examples, emission can be in the infrared (IR) wavelengths from about 700 nm to about 1 mm. In an unlimited example, emission can center in the IR with a wavelength of about 10.6 μm. In several examples, emission can be in the near infrared (NIR) wavelengths from about 800 nm to about 2500 nm. In an example, emission can center in the NIR with wavelengths of about 1060 nm and about 1030 nm. In a number of examples, emission can center in the visible wavelengths from about 380 nm to about 750 nm. In an unlimited example, emission can center in the visible with wavelengths of about 530 nm and about 515 nm. In many examples, emission can center in the ultraviolet (UV) wavelengths from about 100 nm to about 400 nm. In an unlimited example, emission can center in the UV with wavelengths of about 355 nm, about 343 nm, about 248 nm, and about 193 nm.

Laser techniques can create various surface topologies of the materials. Various laser techniques can create patterning including (but not limited to) micropatterns and/or nanopatterns on various surfaces including (but not limited to) even (regular or flat) surfaces, and uneven (or irregular) surfaces. As can readily be appreciated, any of a variety of surfaces can be modified as appropriate to the requirements of specific applications in accordance with various examples of the invention. Structures and/or patterns created by the laser techniques in accordance with several instances can be periodic and/or aperiodic. In certain examples, subtractive laser techniques including (but not limited to) ablation can be applied. In a number of examples, additive laser techniques including (but not limited to) polymerization can be used. Examples of laser techniques include (but are not limited to) direct laser writing (DLW) and interference lithography. Both DLW and interference lithography can be used for subtractive and/or additive processes. As can readily be appreciated, any of a variety of laser techniques can be used as appropriate to the requirements of specific applications in accordance with various examples of the invention.

Direct laser writing (DLW) can create patterns with feature sizes ranging from about 1 micron to about 50 microns. Micro-patterning by DLW may have a writing resolution of about 1 μm. DLW processes can be used for areas with a surface area of at least 1 mm². DLW can also create periodic and/or aperiodic patterns in accordance with some instances. In several examples, DLW can create patterns on both even and uneven surfaces. DLW can be also referred to as mask-less lithography.

Interference lithography can create patterns with sizes ranging from nanometers to microns. Nano-patterning and/or micro-patterning by interference lithography can have a writing resolution of less than about 1 μm. Interference lithography processes can be used for areas with a surface area of at least 5 mm². Interference lithography can create periodic patterns in accordance with some instances. Interference lithography can be applied on flat surfaces.

In many examples, DLW and interference lithography processes can be combined to create surfaces with hierarchical patterns. A number of instances show that hierarchical patterns can be multi-dimensional. The hierarchical patterns can have feature sizes from about tens of nanometers to about hundreds of microns.

Various systems, apparatuses, methods, and devices that can promote endothelization, regeneration and/or healing. Certain examples use one or more laser pulses to create a surface topography of materials used in the construction of a prosthetic valve, vascular stent, and/or any other prosthetic device. Some examples use pulsed lasers including (but not limited to) ultra-short pulse (USP) laser to modify components and/or materials used in the construction of a prosthetic valve or stent. Material modification with USP lasers can be a single-step and contactless method for surface micro-patterning. USP lasers can be used for “cold ablation” and micromachining where melting and heat effects may be detrimental to the materials or where post processing may need to be avoided. Some examples use USP laser ablation to create microgrooves with varying geometries and patterns.

Various laser techniques can be used to modify soft materials including (but not limited to) skirts, textile skirts and leaflets. Laser techniques for soft materials can include (but are not limited to) UV lasers in accordance with some examples. UV lasers can be single wavelength or can be a narrow band with wavelengths ranging from about 100 nm to about 400 nm. Certain examples use UV lasers with a wavelength of about 360 nm.

Laser techniques can be used to modify hard and/or metallic materials utilized in various devices including (but not limited to) stents and frames. Examples of metallic materials used for stents and frames include (but are not limited to) nitinol, Co—Cr alloy, and stainless steel. Laser techniques for hard materials can include (but are not limited to) infrared (IR) and near infrared (NIR) lasers in accordance with some examples. NIR lasers can be single wavelength or can be a narrow band with wavelengths ranging from about 780 nm to about 1060 nm. IR lasers are single wavelengths centered around 10 μm.

Various laser techniques can modify surface topography of a material. Some examples include that surface textures can include one or more pores possessing the size and shape of endothelial cells to encourage endothelial cell implantation (or colonization), growth, and/or reproduction. Additional examples alter surface chemistry to encourage endothelial cell implantation (or colonization), growth, and/or reproduction. The one or more laser pulses may alter (e.g., adding, removing, changing, etc.) ionization state, charge, functional group(s), antigen(s), and/or any other chemical change to the surface of the material. In some examples, the patterning can provide lubricous surfaces to allow for easier manipulation, movement, and/or navigation of the device (e.g., repair device and/or delivery device) to its proper position (e.g., valvular position, position in blood vessel, etc.). Certain examples provide patterning can reduce fibrotic responses, while other examples of patterning may increase fibrotic responses. Several examples provide devices with a combination of patterning, where different characteristics may be desirable at various positions of components of a device, as will be elaborated further herein.

Systems and methods for modifying material properties and structures with laser techniques that can be utilized in the biomedical devices in accordance with various examples of the invention are discussed further below.

Material Modification with Laser Techniques

Systems and methods of laser assisted processes to modify prosthetic devices and/or cardiac repair devices to modify mechanical properties and/or biocompatibilities are described. Many examples show that laser techniques can modify physical properties and/or chemical properties of various materials that constitute biomedical devices. Several examples show that laser assisted processes can modify properties including (but not limited to) topography, surface patterning, mechanical property, chemistry, and surface chemistry, of materials including (but not limited to) soft materials, hard materials, biological materials, and synthetic materials. Laser assisted modification shown in many examples can improve biocompatibility of the materials including (but not limited to) accelerating endothelization, improving cell adhesion, and modulating cell functions.

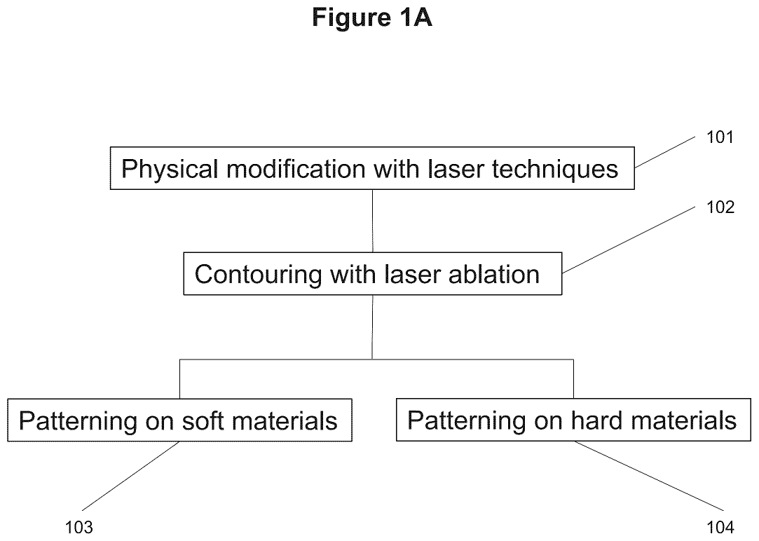

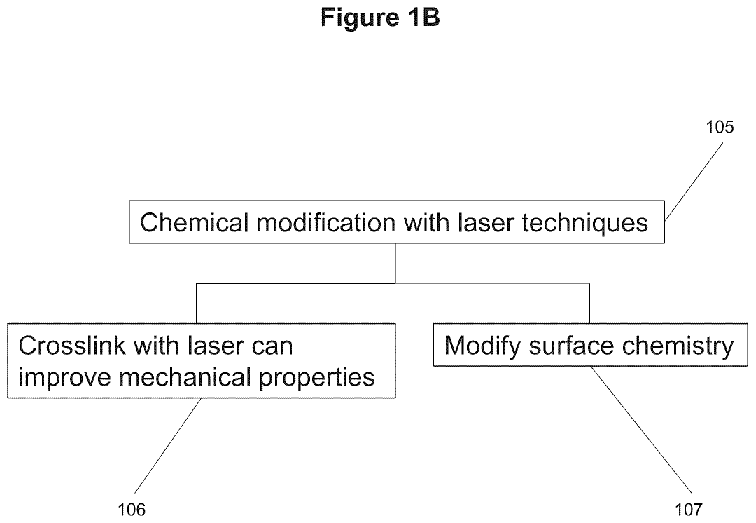

Turning to FIG. 1A, a process of physical modification with laser techniques in accordance with an example of the invention is illustrated. Various laser techniques can be used for physical modification 101. Examples of laser techniques include (but are not limited to) direct laser writing (DLW) and interference lithography. Both DLW and interference lithography can be used for subtractive and/or additive processes. In some examples, subtractive laser techniques including (but not limited to) ablation can be applied. In certain examples, additive laser techniques including (but not limited to) polymerization can be used.

Various laser techniques can modify surface topology and create micropatterns and/or nanopatterns on various surfaces. The surfaces can be even (regular or flat) surfaces, and/or uneven (or irregular) surfaces. Structures and/or patterns created by the laser techniques in accordance with several instances can be periodic and/or aperiodic.

In several examples, laser techniques can modify the thickness and/or structures of various materials. Thickness of a material can be modified using laser ablation 102. Laser ablation in accordance with some examples can be applied to different locations of a material to create different thickness as desired. Contouring with laser ablation in accordance with many examples can provide localized changes and/or gradient changes to material thickness. In certain examples, laser ablation may employ pulsed lasers to remove materials from a substrate for generating micro-structures and/or nano-structures. The pulsed lasers may have pulse widths ranging from milli seconds to sub-pico seconds. Various light sources can be employed for ablation in medical devices. Examples of light sources include (but are not limited to) Nd:YAG, excimer, CO₂, and fiber. Emission wavelengths can center in various ranges of the optical spectrum including (but not limited to) the infrared (IR) wavelengths, the near infrared (NIR) wavelengths, the visible wavelengths, and the ultraviolet (UV) wavelengths.

Many materials used in medical devices can be subjected to laser ablation. Laser ablation can create contouring on soft materials 103 and/or hard materials 104. Examples of soft and hard materials include (but are not limited to) medical grade polymers, medical grade metals and metal alloys, and materials from biological sources. Medical grade polymers can include (but not limited to) polymethylmethacrylate, polyetherketone, polyimide, polyamide, polyethylene, polytetrafluorethylene, nylon, polydimethylsiloxane, silicone, polyethylene terephthalate (PET), polybutylene terephthalate (PBT), polyester, polyurethanes, blocked copolymer of polycarbonate, poly(sulfone of bisphenol-A) (PSU)-PBT copolymers. Medical grade metals can include (but not limited to) nitinol. Materials from biological sources can include (but not limited to) collagen, acrylate collagen composite, chitosan, and pericardium tissue. In several instances, the materials are part of cardiac repair and regenerative devices including (but not limited to) heart valves, prosthetic heart valves, transcatheter heart valves, mitral heart valves, aortic valves, prosthetic valves, stents, vascular stents, patches, vascular patches, and cardiac patches. In certain instances, the materials can be used to make components of a cardiac repair and regenerative device including (but not limited to) sutures, leaflets, prosthetic tissues, skirts, outer skirts, textile skirts, stents, and frames.

Turning to FIG. 1B, a process of chemical modification with laser techniques in accordance with an example of the invention is illustrated. Various laser techniques can be used for chemical modification 105. In several examples, laser modification can provide controlled thermal properties and/or mechanical properties throughout the materials 106. Examples of mechanical properties include (but are not limited to) fatigue resistance, tear propagation rates, ultimate tensile strength, and Young's modulus. Mechanical properties can be modified by adding non-toxic chemical reagents including (but not limited to) photoactive or photo-reagent crosslinkers to various materials. UV light laser can be used to create reactive species and form new chemical bonding in the materials. Examples of the materials include (but are not limited to) soft materials, polymers, biomaterials, proteins, polypeptides, polysaccharides, collagens, chitosan, silk, wool, cellulose, starches, pectin, gelatin, alginates, materials from biological sources, and pericardium tissues. Mechanical properties modification can be achieved by laser assisted coating techniques including (but not limited to) laser alloying. In laser alloying, two materials can be attached or blended together by using laser. Laser assisted crosslink processes can enable precision crosslink of the materials at the desired locations. In some instances, laser techniques can enable localized and/or gradient crosslink of collagen matrix to modify mechanical properties.

In some instances, laser processes can be used to alter surface chemistry 107. Surface chemical properties that can be modified with laser techniques include (but are not limited to) functional groups, ionization, moieties, and/or other chemical makeup. Laser techniques can be used to apply coating layers to at least one component of cardiac repair devices. Surface coating may change hydrophilicity and/or hydrophobicity of the substrate. In some instances, biopolymers and/or biomaterials can be modified using laser assisted coating of hydrophilic polymers.

While various processes for modifying materials using laser techniques are described above with reference to FIGS. 1A and 1B, any variety of processes that utilize laser techniques to modify properties can be utilized in the biomedical devices as appropriate to the requirements of specific applications in accordance with various examples of the invention. Processes for patterning with laser techniques in accordance with various examples of the invention are discussed further below.

Patterning with Laser Techniques

Various methods can modify prosthetic devices and/or cardiac repair devices to promote endothelization. Several examples show patterning with laser direct write and/or interference lithography. Numerous examples utilize lasers including (but not limited to) ultra-short pulse lasers to create patterning on such devices. Laser patterning can be applied to soft and/or hard materials. Patterning with laser in accordance with many examples can be carried out on even, uneven, flat, and/or curved surfaces.

Cells can be highly influenced by surface topography, including influencing their cell size, shape, adhesion, migration, and/or proliferation. Surface topography with sub-micrometer dimensions can affect cell adhesion and cellular function in addition to modulating cell-cell interactions. Furthermore, specific surface textures and patterns can encourage endothelization and/or promote anti-coagulation. (See e.g., Liliensiek SJ, et al. Modulation of human vascular endothelial cell behaviors by nanotopographic cues. Biomaterials 2010; 31 (20): 5418-5426; Zheng N, et al. Preparation of micro-patterned surfaces of Si—N—O films and their influence on adhesion behavior of endothelial cells. Sci China Tech Sci 2010; 53 (1): 257-263; Franco D, et al. Control of initial endothelial spreading by topographic activation of focal adhesion kinase. Soft Matter 2011; 7:7313-7324; Dickinson L E, et al. Endothelial cell responses to micropillar substrates if varying dimensions and stiffness. J Biomed Mater Res 2012; 100A (6): 1457-1466; and Aktas C, et al. Micro- and nanostructured Al₂O₃ surfaces for controlled vascular endothelial and smooth muscle cell adhesion and proliferation. Mater Sci Eng C 2012; 32:1017-1024; the disclosures of which are hereby incorporated by reference in their entireties.)

Numerous instances provide processes and methods to create patterns that can improve, accelerate, and/or encourage healthy reendothelization. In various instances, microscale patterns with different dimensions and/or geometries can be created on materials including (but not limited to) metals, metal alloys, ceramics, plastics, and/or thin films by varying one or more laser parameters. Laser parameters including (but not limited to) excitation wavelength, repetition rate, energy per pulse, and overlapping distance between pulses, can be varied to optimize groove quality and precision in accordance with several examples. Depending on the specific materials being patterned, laser parameters such as energy per laser pulse can be changed to adapt to the processes. In DLW, ablated grooves in polymer substrates can be formed with increasing depth profiles by increasing laser powers, while other parameters can be kept constant. In one unlimited example, laser ablation can be carried out with PMMA using USP lasers. The laser parameters used in the ablation of PMMA can include about 1030 nm of wavelength; about 240 fs pulse duration; about 610 kHz in frequency; about 1 μJ/pulse in energy; about 2 mm/s in scanning rate.

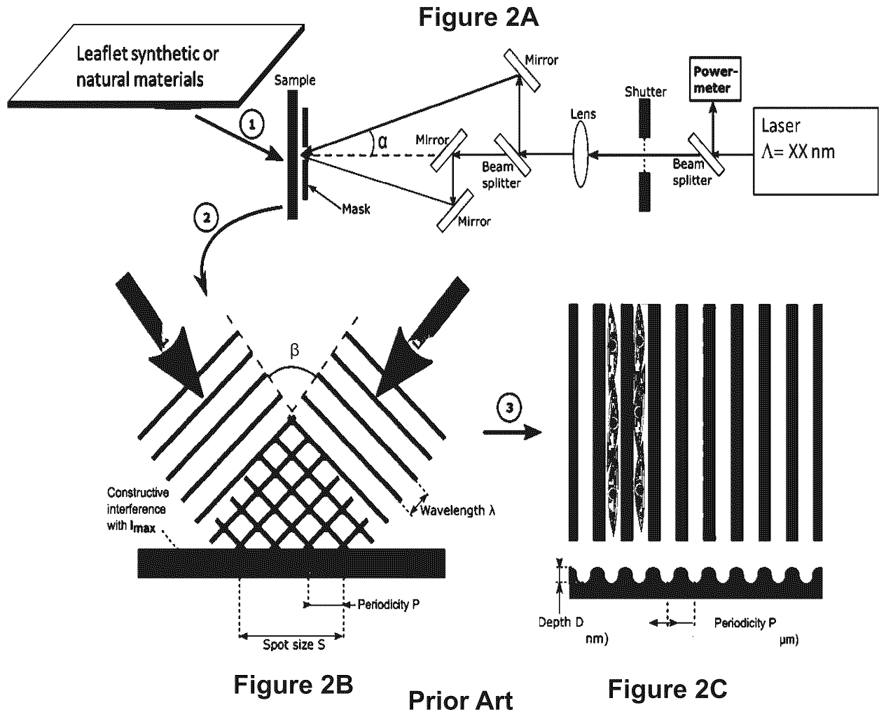

Various instances utilize one or more of interference lithography and direct laser writing to create patterns on surfaces. Turning to FIG. 2A, an exemplary laser system for interference lithography is illustrated. Interference lithography is typically used to create regular or periodic patterns on flat surfaces and can generally be used over a wide range of sizes, from approximately 100 nm and larger. In accordance with various examples, a laser system for interference lithography comprises a laser source generates a beam with a suitable wavelength for deposition or ablation, depending on material and desired patterning. In some instances, the beam passes through a beam splitter to route two beams which intersect at an angle to create an interference pattern. In certain aspects, one beam is routed a distance, such that one beam is offset by a distance of ½ the wavelength of the beam.

Turning to FIG. 2B, the intersecting beams create an interference pattern on the material to be patterned. Specifically, the two beams intersect at angle β to create interference of the laser beams. In certain instances, positive interference of the beams increases the power at specific positions to allow for ablation of the material. In other instances, the negative interference of the beams reduces the power of the beams to prevent ablation of the material to be patterned. The specific distance between the interference creates a spatial period (or periodicity) in the material. This periodicity is a function of angle β and the wavelength of the laser. It should be noted that while the above example describes subtractive methods, similar applications can be used for additive methods, such that positive interference allows for deposition of material or negative interference prevents deposition of material.

FIG. 2C illustrates an exemplary patterning generated by an interference lithography system, where the interference pattern created by the intersecting beams generates the troughs in the material to be patterned at the specific periodicity.

Returning to FIG. 2A, additional features of the laser system include an additional beam splitter to route the beam to a power meter to monitor laser strength. Certain instances further include a power regulator to alter laser strength, manually and/or through an automatic feedback given readings from a power meter. Some instances include a shutter and/or aperture to influence the beam width or ability to transmit through the system. Additional instances include one or more lenses to assist in focusing and/or routing a beam. Such lenses can be located at any position to provide the proper routing or focusing of the beam. Furthermore, polarizers and retarders are used to ensure the proper light polarizations of the two beams at the interference location.

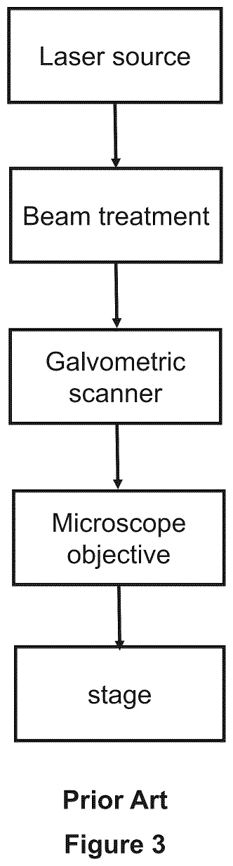

Turning to FIG. 3, an exemplary system for direct laser writing is illustrated. Such systems include a laser and optics to direct the laser beam to a stage. In many instances, the stage is where the material to be patterned is located. In various instances, the stage can be moved in one or more directions (e.g., X-axis, Y-axis, Z-axis, rotational around one or more axes, etc.). In some instances, the laser beam can be moved to pattern material located on the stage. The patterns on the substrate can be created by moving the laser beam on a fixed substrate using galvaomteric mirrors, and/or moving the substrate around a fixed laser beam using stages. The laser beam and the substrate can be moved simultaneously. The optics used in laser direct writing can be a focusing optic including (but not limited to) a microscope objective or an f-theta lens. Direct laser writing is known to produce patterns with feature sizes ranging from approximately 1 μm to approximately 50 μm. Direct laser writing also allows for custom patterns, including periodic and/or aperiodic patterning on material. Additionally, patterning created by direct laser writing can include subtractive (e.g., laser ablation) or additive (e.g., laser sintering) methods to a material to be patterned.

Line-patterning may influence the regeneration of a healthy endothelial cell monolayer of human vascular vein endothelial cells and human cardiac microvascular endothelial cells. (See e.g., Ding Y, et al. Directing vascular cell selectivity and hemocompatibility on patterned platforms featuring variable topographic geometry and size. ACS Appl Mater Interfaces 2014; 6:12062-12070; and Pacharra S, et al. Surface patterning of a novel PEG-functionalized poly-L-lactide polymer to improve its biocompatibility: Applications to bioresorbable vascular stents. J Biomed Mater Res B Appl Biomater. 2019; 107 (3): 624-634; the disclosures of which are hereby incorporated by reference in their entireties.) Many instances modify various surfaces with line patterning to improve biocompatibility. Certain instances use patterns of pores to promote anti-coagulation. In various instances, pores can have various shape including (but not limited to) circular, rectangular, square, hexagonal, octagonal, and/or any other polygonal shape. As can readily be appreciated, any of a variety of shapes can be utilized as appropriate to the requirements of specific applications in accordance with various examples of the invention. Several instances provide subtractive and/or ablative processes (removal of materials) with laser techniques to create the patterns. Many instances can create the patterning by deposition of material (e.g., additive methods of manufacture). Patterns created using additive laser processes include (but are not limited to) elevated pillars, columns, and any elevated shapes.

The patterning can create multi-dimensional structures and/or patterns including (but not limited to) two dimensional (2D), three dimensional (3D), 2D and 3D, that may aid in reendothelization. Several instances provide the structures and/or patterns can include various shapes including (but not limited to) groves, pillars, pores, ridges, waves, and/or any other pattern shown to have the desired biologic effect including (but not limited to) endothelization and healing. As can readily be appreciated, any of a variety of patterns can be utilized as appropriate to the requirements of specific applications in accordance with various examples of the invention. Some instances provide the surfaces can be patterned with periodic and/or random structures including (but not limited to) grooves, dimples, and squares that can enhance cellular adhesion, migration and proliferation. Many instances provide at least one dimension of the patterns and/or structures can range from microscale (from about 1 μm to about 999 μm) to nano-scale (from about 10 nm to about 999 nm). Certain instances provide that the patterns on materials may enable the material to be anisotropic.

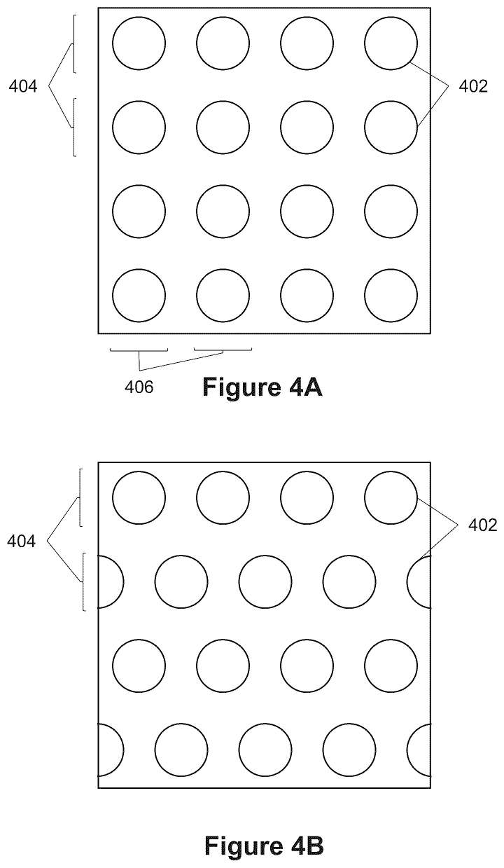

Micropatterns can be created on soft materials including (but not limited to) leaflets and skirts of cardiac repair devices. Turning to FIG. 4A, various examples show individual pores 402 in a series of rows 404 and columns 406, where the rows can be parallel to every other row and every row, each column can be parallel to every column, and every row can be perpendicular to every column. However, as seen in FIG. 4B, some examples provide pores 402 in a series of parallel rows 404, where neighboring rows can be offset, such to form a honeycomb pattern. While FIGS. 4A-4B illustrate round or circular pores, such pores are merely representative of the overall pattern. As such, pores in various examples can be any geometry to promote, encourage, and/or enhance endothelization, including (but not limited to) circular, ovular, oblong, triangular, quadrilateral (e.g., square, rectangular, rhomboidal, trapezoidal), hexagonal, octagonal, any other regular or irregular shape or polygon, and combinations thereof.

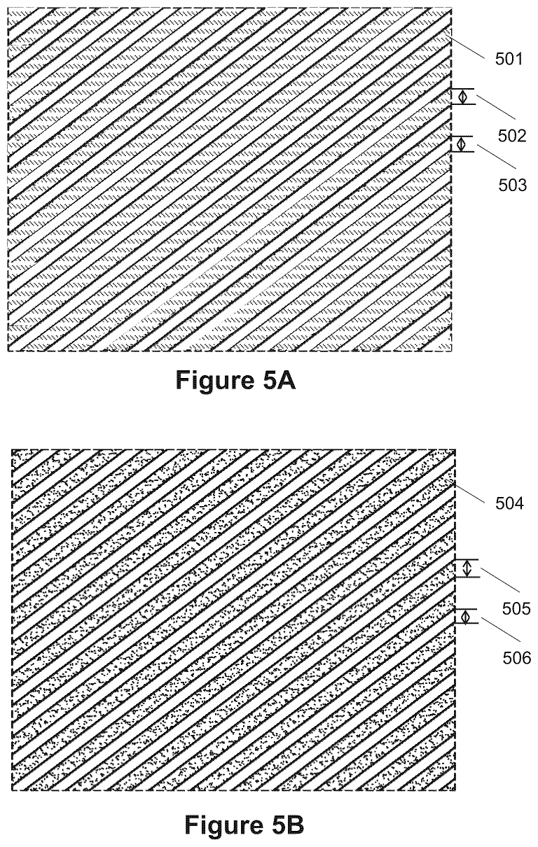

Additional examples of patterning on soft materials can include parallel rows, such as illustrated in FIGS. 5A-5B. Such rows can be created by ablation or subtraction of the material, e.g., troughs or trenches (e.g., FIG. 5A) or deposition or addition of material, creating ridges on the material (e.g., FIG. 5B). The dimensions between such rows can be of any suitable height (or depth), width, and spacing suitable for encouraging, enhancing, and/or promoting endothelization. In ablated examples, such as troughs 501 illustrated in FIGS. 5A, the depth ranges from about 1 μm to about 10 μm, while the width of the troughs 502 from about 5 μm to about 20 μm is suitable for cellular implantation. The spacing between troughs 503 can vary to allow cellular growth and/or allow for flexibility of the underlying material, and can be between about 5 μm and about 20 μm. Additionally, in deposited examples, such as ridges 304 illustrated in FIGS. 5B, the height of such ridges ranges from about 1 μm to about 10 μm, while the width of the ridges 505 from about 1 μm to about 5 μm is suitable for cellular implantation. Spacing between ridges 506 can vary to allow cellular growth and/or allow for flexibility of the underlying material, and can be between about 5 μm and about 20 μm. While FIGS. 5A-5B illustrate linear rows, various examples provide zigzags, waves, sinusoids, and/or another other patterning of trenches and/or ridges on the material that promotes, encourages, or enhances endothelization, and/or provides desired properties (e.g., rigidity, flexibility, etc.) to the underlying material.

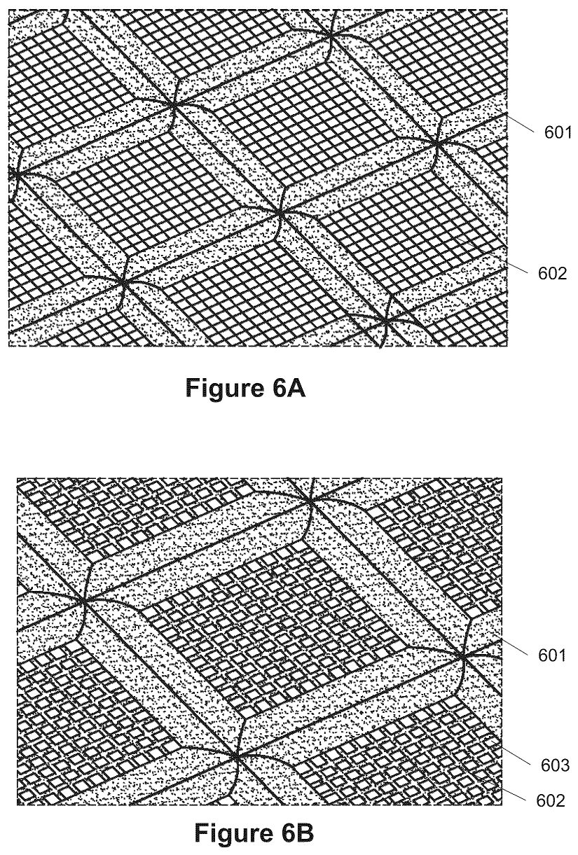

Further examples create materials with multidimensional and/or multilayered patterning. Some instances can create smaller patterns (e.g., nanopatterns) within a larger size pattern (e.g., micropattern). An example of such patterning is shown in FIGS. 6A-6B. FIG. 6A illustrates a “chessboard” pattern, where a larger grid 601 is created over a smaller grid 602. The height of the larger grid 601 can be taller than the smaller grid 602. The smaller grids 602 can be embedded into the larger grids 601. The grid lines of the larger grid can protrude from the surface. The larger grids and the smaller grids can be square or rectangular shapes. FIG. 6B illustrates an enlarged single smaller grid of FIG. 6A. Each square of the grid 603 created by the grid lines of the smaller grid 602 can protrude from the surface. The dimensions of the patterns can be of any suitable height (or depth), width, and spacing suitable for encouraging, enhancing, and/or promoting endothelization. In the smaller grids 602, the depth of the protruding grids can range from about 10 nm to about 50 μm; up to about 40 μm; up to about 30 μm; and up to about 20 μm, while the width of the grids from about 10 nm to about 50 μm; up to about 40 μm; up to about 30 μm; and up to about 20 μm; is suitable for cellular implantation. The spacing between the grids can vary to allow cellular growth and/or allow for flexibility of the underlying material. In various instances, the larger grid can be created via direct laser writing or interference lithography, while the smaller pattern is created via interference lithography. Direct laser writing can create patterns on even and/or uneven surfaces. Interference lithography may be able to create patterns on even surfaces. While FIGS. 6A-6B illustrate square shapes of the multidimensional structures, such shapes are merely representative of the overall pattern. As such, multidimensional structures in various examples can be any geometry to promote, encourage, and/or enhance endothelization, including (but not limited to) circular, ovular, oblong, triangular, quadrilateral (e.g., square, rectangular, rhomboidal, trapezoidal), hexagonal, octagonal, any other regular or irregular shape or polygon, and combinations thereof.

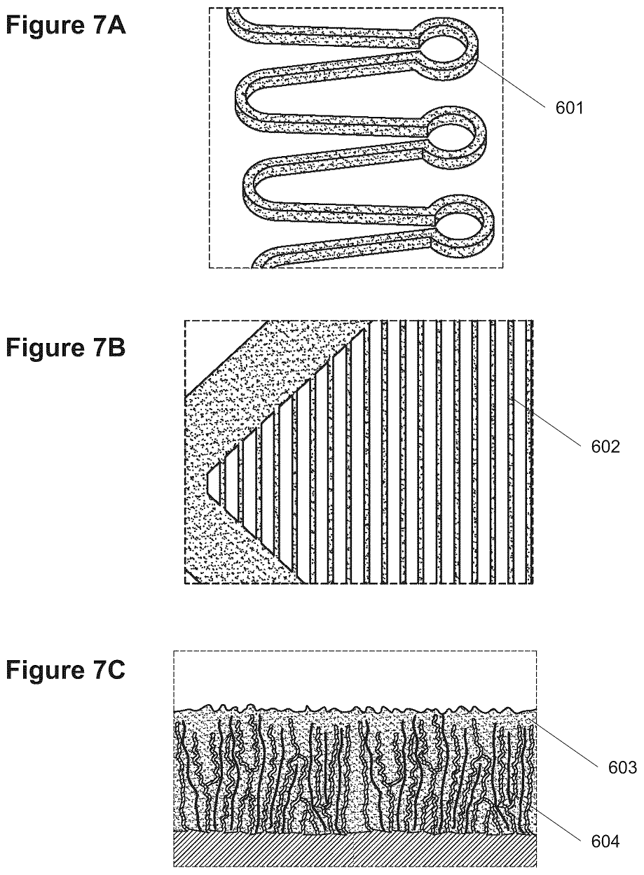

Examples of patterns on hard materials including (but not limited to) metals, metal alloys, nitinol, Co—Cr alloy, and stainless steel, can include (but are not limited to) curves, straight lines, wrinkled lines, parallel rows, geometrical shapes, squares, rectangular, and circles. The patterns on the hard materials can be created by ablation or subtraction of the material, or deposition or addition of material, creating ridges on the material. Interference lithography and/or direct laser writing processes can be used to create various patterns on metal surfaces. Specific laser parameters can be tuned for metal surfaces. In some instances, infrared laser may be used for metal surfaces. Interference lithography and/or direct laser writing processes may be applied to subtractive processes and/or laser ablation processes. In certain instances, electroforming and/or laser sintering can be used for modifying metal surfaces. Ti sapphire femtosecond lasers and/or Ti sapphire lasers can be used to modify the surfaces of metal materials such as nitinol. In some examples, laser patterning and/or machining of the nitinol stent can create nanogrooves and/or microgrooves on outside surface of the stent to allow for an easier tissue ingrowth to prevent paravalvular (PVL) leaks. Examples of surface patterning with laser techniques on nitinol are shown in FIG. 7A-7C. FIG. 7A illustrates microgroove patterns on nitinol surfaces created by laser. The microgrooves 701 can be protruding from the surface and created by additive laser processes. The microgrooves can otherwise be created using ablative laser processes by removing materials from the nitinol surfaces. Alternatively, the microgrooves can be created using additive processes, where functional materials including (but not limited to) materials with pharmacological effects may be added to the surface. The microgrooves can be created in a continuous line, either straight or curved. The microgrooves can have various shapes including (but not limited to) circles. The depth of the microgrooves can vary from about 1 μm to about 100 microns. FIG. 7B illustrates microgroove patterns on nitinol surfaces created by laser. The microgroove patterns 702 can be parallel straight lines. The width of the microgroove can vary from about 10 nm to about 100 microns. The space between the microgrooves can vary from about 500 nm to about 100 microns. FIG. 7C illustrates surface pattern on a nitinol surface after laser treatment. The nitinol surface 603 can be a flat surface or a curved surface (e.g., a rod). Irregular wrinkled patterns 604 can be created on nitinol surfaces with laser treatment. The patterned lines may not be parallel and may not be straights. While FIGS. 7A-7C illustrate linear rows, various examples provide zigzags, waves, sinusoids, and/or another other patterning of trenches and/or ridges. As such, patterns on metal surfaces can be any geometry to promote, encourage, and/or enhance endothelization.

While various processes for patterning materials using laser techniques are described above with reference to FIGS. 4-7, any variety of patterns that can be created using laser techniques to improve biocompatibility can be utilized in the biomedical devices as appropriate to the requirements of specific applications in accordance with various examples of the invention. Processes for surface modification with laser ablation in accordance with various examples of the invention are discussed further below.

### Laser Ablation

In certain examples, laser assisted processes can modify the thickness and/or structures of various materials. Thickness of a material can be modified by laser ablation, which may employ pulsed lasers to remove materials from a substrate for generating micro- and/or nano-structures. Many materials used in medical devices can be subjected to laser ablation. Laser ablation can create contouring on various materials including (but not limited to) medical grade polymers, medical grade metals, and materials from biological sources. Examples of medical grade biodegradable polymers include (but are not limited to) glycolide-based copolymers, lactide-based copolymers, poly(lactic-co-glycolic) acid, poly(α-hydroxy acids), caprolactone-based polymers, cross-linked polyester hydrogels, poly(orthoesters), poly(glycerol-co-sebacate) (PGS), polyaniline (PANI), polypyrrole (PPy), poly(3,4-ethylenedioxythiophene) (PEDOT), polyanhydrides, polyethylene glycol, poly(vinyl alcohol) (PVA), albumen, melanin, dioxanone-based polymers. Examples of medical grade biostable polymers include (but are not limited to) polyacrylates, polyether, poly(styrene-b-isobutylene-b-styrene), polysulfones, polyethersulfones, polymethylmethacrylate, polyetherketone, poly(vinylidene fluoride) polyimide, polyamide, polyethylene, polytetrafluorethylene, nylon, polydimethylsiloxane, silicone, polyethylene terephthalate, polyester, polyurethanes, polystyrene, and polyvinyl chlorides. Examples of medical grade metals include (but are not limited to) nitinol. Examples of materials from biological sources include (but are not limited to) collagen, chitosan, and pericardium tissue.

Many polymers used in medical devices can be subjected to laser ablation. In many examples, pulsed lasers may provide a high efficiency in material removal. The pulsed lasers may have pulse widths ranging from milli seconds to picoseconds to femtoseconds. Various laser sources including (but not limited to) neodymium-doped yttrium aluminum garnet (Nd:YAG), excimer, carbon dioxide (CO₂), and fiber can be employed for ablation in medical devices.

Emission wavelengths can center in various ranges of the optical spectrum. In some examples, emission can be in the infrared (IR) wavelengths from about 700 nm to about 1 mm. In an unlimited example, emission can center in the IR with a wavelength of about 10.6 μm. Examples of lasers emitting IR light can include the carbon dioxide (CO₂) lasers. The center wavelength of the CO₂ laser is around 10.6 μm, and it can be modulated to produce microsecond long pulses with large laser average power. Because of their emission wavelength, IR lasers can be used for material ablation following a photothermal process. Infrared lasers can also be used for ablation and surface modification of materials. For example, an infrared laser can be used in surface texturing and ablating of PTFE and polyimide materials.

In several examples, emission can be in the near infrared (NIR) wavelengths from about 800 nm to about 2500 nm. NIR lasers can produce sub-picosecond pulses at high repletion rates (in the range of MHz), thus delivering efficient ablations with large removal rates. Emission wavelengths of NIR lasers can be about 800 nm, about 1030 nm, and about 1050 nm. Because pulsed NIR lasers can achieve high light intensities, substrate ablation with NIR lasers may occur via a photophysical processes. In an example, emission can center in the NIR with wavelengths of about 1060 nm and about 1030 nm.

In a number of examples, emission can center in the visible wavelengths from about 380 nm to about 750 nm. In an unlimited example, emission can center in the visible with wavelengths of about 530 nm and about 515 nm. Visible wavelength lasers used in material ablation can be harmonics of NIR lasers. In some examples, the frequency doubled Nd-YAG laser can produce nanosecond log pulses centered around 532 nm that are effective in the subtractive modification of different materials.

In many examples, emission can center in the ultraviolet (UV) wavelengths from about 100 nm to about 400 nm. Due to the emission wavelengths and pulse energies, UV lasers such as excimer lasers can deliver substrate ablation by a photochemical process. In an unlimited example, emission can center in the UV with wavelengths of about 355 nm, about 343 nm, about 248 nm, and about 193 nm. Examples of UV lasers include the ones based on the formation of excimer species in the gas phase. Examples of excimer lasers include the ArF, and the KrF, with emission wavelengths centered around 193 nm and around 248 nm, respectively.

NIR lasers with sub-picosecond pulses and their harmonics in the visible and UV regions of the optical spectrum are known as ultra-short pulsed (USP) lasers. Ultra-short pulsed (USP) lasers can be used for “cold ablation” and micromachining where melting and heat effects may be detrimental to the material or where post processing may need to be eliminated. USP lasers can work on various types of materials and can be easily adapted and scaled up for micropatterning of complex component shapes. In many examples, USP laser ablation can be used to create microgrooves with varying geometries and patterns. Such patterning in accordance with several examples can allow for tissue ingrowth, healing, regeneration, endothelization, and/or any other biological phenomena that can increase the likelihood of implant acceptance.

Various laser sources can be chosen based on the materials. In some examples, laser sources including (but not limited to) KrF, Nd:YdYAG, ArF, KrCl, XeCI, and/or CO₂, can be used for PMMA laser treatment. In several examples, laser sources including (but not limited to) XeCI, KrF, and/or Nd:YAG can be used for polyetherketone laser treatment. In certain examples, laser sources including (but not limited to) XeCI, KrF, ArF, and/or XeF can be used for polyimide and/or polyamide laser treatment. In a number of examples, laser sources including (but not limited to) iodine PALS can be used for polyethylene laser treatment. In several examples, laser sources including (but not limited to) KrF can be used for PTFE laser treatment. In some examples, laser sources including (but not limited to) XeCI can be used for nylon laser treatment. In certain examples, laser sources including (but not limited to) CO₂ laser, Ti sapphire laser, ArF laser, and/or erbium doped fiber laser can be used for PDMS and/or silicone laser treatment. In a number of examples, laser sources including (but not limited to) ArF, KrF, and/or XeCI can be used for PET and/or polyester laser treatment. In several examples, laser sources including (but not limited to) Ti sapphire femtosecond laser can be used for nitinol laser treatment. In various examples, UV lasers (wavelength from about 100 nm to about 400 nm) with laser sources including (but not limited to) Nd:YAG, fiber lasers, and/or excimer lasers, can be used for laser treatment of biological materials, biological tissues, collagen, chitosan, and/or pericardium tissues.

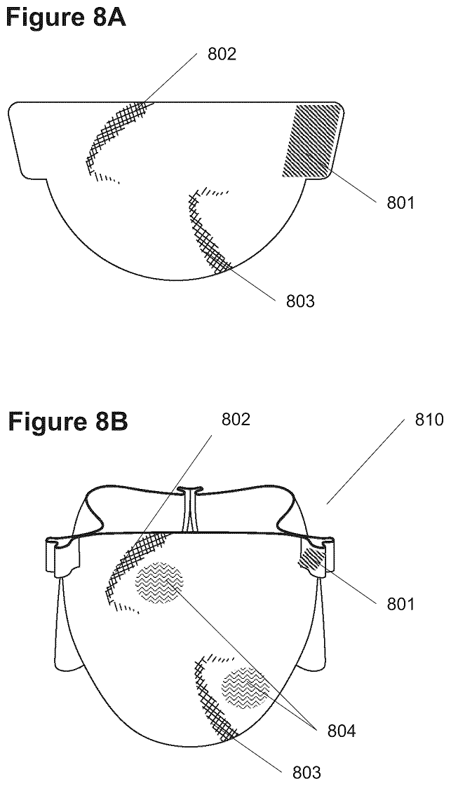

Laser ablation can be used to create various thickness throughout the materials. In many examples, laser ablation can be used to contour a surface to achieve desired thickness at specific areas. In certain examples, laser assisted crosslink can improve mechanical properties of specific areas where thickness has been reduced. In several examples, laser ablation can be used on pericardium tissues to modify thickness from about 500 microns to about 150 microns. In certain examples, laser ablation can be used to create various patterns on pericardium tissues to improve biocompatibility. Turning to FIG. 8A-8B, examples of laser ablation on pericardium tissue are illustrated. In FIG. 8A, laser ablation can be applied to modify the pericardium tissue before and/or after the tissue is cut into shape. The edges of the pericardium tissue 801 can be thicker. Laser ablation can be applied to selected areas 802 to even out the thickness throughout the pericardium tissue to achieve thickness consistency. The thickness of the pericardium tissue can be from about 100 microns to about 500 microns. Laser ablation can be applied to selected areas 803 to create micropatterns and/or nanopatterns on the pericardium tissue to modify biocompatibility to promote, encourage, and/or enhance endothelization.

Laser techniques can be combined to create desired thickness and mechanical properties for leaflets in accordance with many examples. Laser ablation can efficiently remove materials to modify the thickness, while laser assisted crosslink can reinforce the mechanical properties of the thinned materials. FIG. 8B illustrates the combination of laser ablation and laser assisted crosslink on a leaflet of a heart valve. Pericardium tissues can be connected to form the leaflet of a heart valve 810. Laser modification can be carried out before and/or after a leaflet is being fabricated. The edges of the pericardium tissue 801 where the tissues are connected can be thicker. Laser ablation may be applied to reduce thickness at various areas 802, and to create patterns at selected areas 803. Laser assisted crosslink can be carried in or near those areas 804 to reinforce the mechanical strength of the leaflet.

While various processes for surface modification using laser ablation are described above with reference to FIGS. 8A-8B, any variety of processes that utilize laser ablation to alter material properties can be utilized in the biomedical devices as appropriate to the requirements of specific applications in accordance with various examples of the invention. Processes for laser assisted crosslinking in accordance with various examples of the invention are discussed further below.

Crosslinking with Laser Techniques

One major challenge of implantable prostheses (such as prosthetic heart valve, vascular graft) constructed with animal derived pericardial tissues can be the tissue durability in vivo. Tissue durability in vivo can be improved by changing tissue chemistry via crosslinking, for example glutaraldehyde (GA) or 1-ethyl-3-(3-dimethylaminopropyl) carbodiimide (EDC). However, when using chemical agents as crosslinker, residual of unreacted chemicals and/or chemical byproducts are often left behind. The residues may need to be washed out to minimize cytotoxicity. Many instances provide materials and systems that can form artificial crosslinking bonds in tissue in situ without the use of any toxic chemicals such as GA. Additionally, animal derived tissues may have variable thickness and mechanical properties. Several instances show that laser treatments including (but not limited to) laser ablation and laser assisted crosslink can modify geometrical dimensions and improve mechanical properties of various devices.

Laser induced crosslinking of proteins, including bovine serum albumin hyaluronate and collagen has been previously reported. (See, e.g., S. Shavkuta, et al, Laser Physics Letter, 2018; 15:015602; U.S. patent application No. 20180193188A1 to S. Vukelic et al.; Sheldon J.J. et al., Optica. 2016; 3 (5): 469-472; the disclosures of which are incorporated herein by references.) Many instances provide laser techniques including (but not limited to) picosecond and/or femtosecond lasers in the wavelength range of NIR from about 500 nm to about 700 nm to create covalent bonds between intermolecular collagen structures. The effectiveness of laser treatment may depend on the sample preparation, laser frequency and laser power in accordance with instances. At a higher power, femtosecond laser may be able to penetrate deeper into thicker collagen structures. Several instances provide that cytotoxic compounds may not be formed using laser crosslinking methods. In some instances, the laser crosslinking processes are generally less aggressive in improving mechanical properties of collagen compared to chemical methods.

For bioprosthetic valves that use bovine or porcine pericardium tissues as leaflets, UV light at about 370 nm in combination with riboflavin (vitamin B2) can increase biomechanical strength of collagen fibers. During laser treatment of the materials in accordance with several examples, additional chemicals can be used to further improve the properties of the material. In some examples, photoactive or thermally active reagents can be incorporated into the material by soaking the material in a solution of the reagent. In certain examples, the photoactive or thermally active reagent can be applied to the surface of the materials by dipping or spray coating processes before the laser treatment. The use of laser and photoactive reagents including (but not limited to) riboflavin can eliminate the use of toxic reagents including (but not limited to) glutaraldehyde during crosslinking processes. Examples of chemical reagents that can be photo/UV or thermally activated by laser include (but are not limited to) aryl azides, azido-methyl-coumarins, benzophenones, anthraquinones, diazo compounds, diazirines psoralen derivatives, vitamin B1 (thiamine), vitamin B2 (riboflavin), vitamin B3 (niacin), vitamin B5 (pantothenic acid), vitamin B6 (pyridoxine), vitamin B7 (biotin), vitamin B12 (cobalamin), folic acid, n-hydroxy succinimide ester of acrylic acid (ANHS), deoxyribonucleic acid, bis-acylphosphine oxides, pyrolipid, porphyrin, chlorin; photopolymerization free radical photo initiators such as 2-hydroxy-4′-(2-hydroxyethoxy)-2-methylpropiophenone (Irgacure 2959 photoinitiator, BASF), methyl phenylglyoxylate; phospholipid-based initiators such as phenyl bis(2,4,6-trimethylbenzoyl) phosphine oxide; sulfo-LC-SDA (Diazirine) and triethylamine. Examples of polymers that can be photo/UV or thermally activated by laser include (but are not limited to) poly [2-methoxy-5-(2-ethylhexyloxy)-1,4-phenylenevinylene MEH-PPV, poly(3-butylthiophene-2,5-diyl), poly(9,9-dioctylfluorene-alt-bithiophene), poly(9,9-di-n-octylfluorenyl-2,7-diyl) (PTO), poly [(9,9-bis(3′-(N,N-dimethylamino) propyl)-2,7-fluorene)-alt-2,7-(9,9-dioctylfluorene)] (PFN-DOF), poly(2,5-di(hexyloxy) cyanoterephthalylidene), poly(5-(2-ethylhexyloxy)-2-methoxy-cyanoterephthalylidene), poly(1,4-phenylene-1,2-ethenediyl) PPV copolymer, poly(9,9-di-(2-ethylhexyl)-9H-fluorene-2,7-vinylene), and poly(2,5-dioctyl-1,4-phenylenevinylene). Thermal crosslinking agents that can be activated by laser include (but are not limited to) curcumin diferuloylmethane. As can readily be appreciated, any of a variety of chemical reagents can be utilized as appropriate to the requirements of specific applications in accordance with various examples of the invention.

The concentrations of the crosslinking reagent mixtures in accordance with several examples may vary between about 5 mM and about 25 mM dissolved in a biologically compatible solvents and/or solutions including (but not limited to) DMSO, PBS saline, and water. In some instances, crosslinkers may be liquid or semisolid at a temperature range from about 10° C. to about 50° C. Liquid crosslinkers can form melts, meaning that they are liquid without the addition of other liquids.

Laser can be used to activate the crosslinking reagents to achieve localized and/or gradient crosslink. UV light lasers can be used to mechanically transform the surface of the materials and modify the chemical structures such that the material may gain new biomechanical properties. Photoactivation of the reagents can be achieved in a wavelength range from about 220 nm to about 400 nm. In many instances, bioprosthetic valves can be soaked in the riboflavin solution to enable the pericardium tissues to absorb riboflavin. In some examples, absorbing wavelength for riboflavin can be about 370 nm with photoactivation time less than about 1 hour.

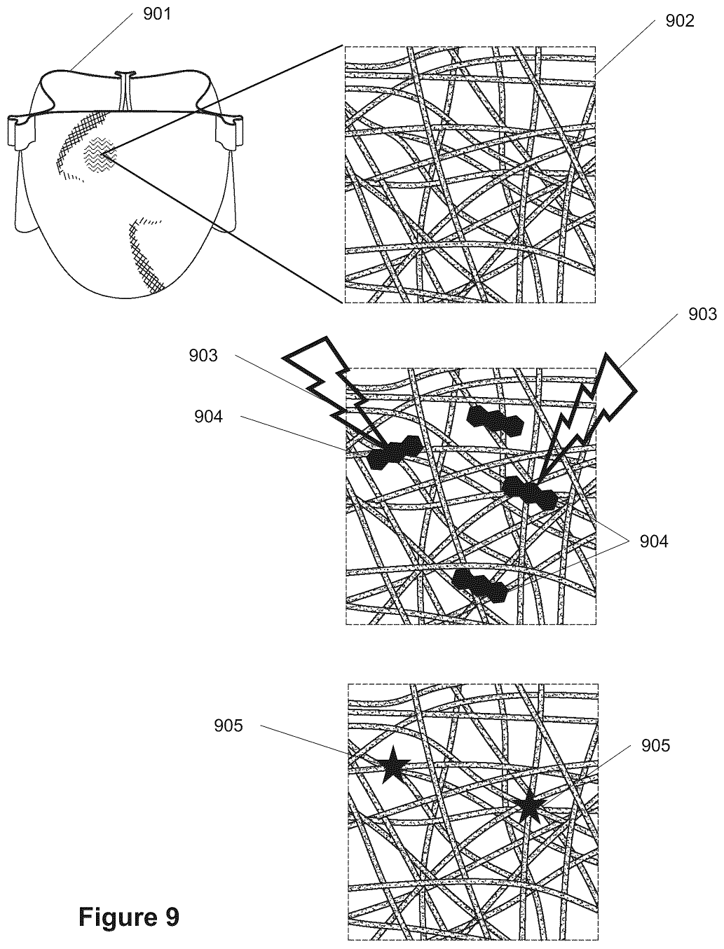

In many examples, UV laser including (but not limited to) wavelength at about 370 nm can be used to focus directly on bioprosthetic valve tissue leaflets to activate photoactive riboflavin molecules and allow for riboflavin to crosslink collagen fibers of the leaflets. FIG. 9 illustrates an example of localized and gradient collagen crosslink with riboflavin using laser. Bioprosthetic valve leaflets 901 can be made with pericardium tissues. Laser assisted crosslink can be applied to the leaflets before and/or after the valve is fabricated. Pericardium tissues can include collagen fibers as one of the components. The collagen fibers may have a matrix-like structure 902. Riboflavin 904 can be added to collagen network 902 prior to the laser treatment. Riboflavin can be incorporated to the collagen by either soaking the pericardium tissue in a solution of riboflavin or applying the riboflavin to the surface of the pericardium tissue. UV laser lights 903 can be used to activate the crosslink process. UV laser lights can be applied directly to the areas of the pericardium tissue where crosslink is desired. Some examples use femtosecond laser pulses. The laser can activate riboflavin 904. Laser crosslinking mechanism includes free oxygen radical (not shown) formation that interact and crosslink, which can stabilize intramolecular and intermolecular collagen structures. Laser assisted crosslink enable localized and gradient crosslink of the tissue 905. Laser enables precision crosslink at the sites where the laser light is applied.

Many instances provide methods of crosslinking biomaterials including (but not limited to) collagen and elastin to improve mechanical properties. The collagen and/or elastin materials can be present in the bovine or porcine pericardium tissue. Crosslinking in accordance with several examples may improve the mechanical properties including (but not limited to) fatigue strength of the pericardial or other tissue types. Mechanical properties modification can be achieved by laser assisted coating techniques including (but not limited to) laser alloying. In laser alloying, two materials can be attached or blended together by using laser.

In several examples, laser treatment can enable more consistent mechanical properties of biological materials. Glutaraldehyde fixation may be able to achieve high mechanical strength. The ultimate tensile strength (UTS) of bovine pericardial tissue after glutaraldehyde fixation process ranges anywhere between about 10 MPa and about 35 MPa. The downside of glutaraldehyde fixation is its poor control and high variability between sample to sample. In many examples, the laser induced crosslinking can not only enable collagen chemical crosslinking without glutaraldehyde, but also provide more consistent mechanical properties, thermal properties, and thickness. Table 1 below lists thickness and various mechanical properties of bovine pericardial tissue with different crosslinking treatment. The bovine pericardial (BP) tissues can be treated by a glutaraldehyde fixation process or laser assisted crosslinking. The laser processed BP tissues show more consistent thickness between about 200 microns and about 250 microns; more consistent UTS between about 20 MPa and 25 MPa; more consistent Young's modulus between about 35 MPa and about 40 MPa; and more consistent shrinkage temperature between about 75° C. and about 76° C., when compared to BP tissue under a glutaraldehyde fixation process. The durability (fatigue) of the laser processed tissue can be tested in accelerated wear testing (AWT) for heart valves. The leaflet with laser processed bovine pericardial passes the 300 million cycles requirement, by which the laser process can maintain the same fatigue resistance of collagen structure of the tissue.

TABLE 1
THICKNESS AND MECHANICAL PROPERTIES
OF BOVINE PERICARDIAL TISSUES.

| | | | | Young's |
|---|---|---|---|---|
| | Thickness | UTS | Shrinkage Temp., | Modulus |
| | (μm) | (MPa) | DSC (° C.) | (MPa) |
| Glutaraldehyde | 250-500 | 10-35 | 79-83 | 35-90 |
| Laser | 200-250 | 20-25 | 75-76 | 35-40 |

While various processes for crosslinking using laser are described above with reference to FIG. 9, any variety of processes that utilize laser techniques to crosslink materials with chemical reagents can be utilized in the biomedical devices as appropriate to the requirements of specific applications in accordance with various examples of the invention. Processes for surface coating using laser techniques in accordance with various examples of the invention are discussed further below.

### Laser Modification of Surface Chemistry

Some instances use at least one laser processes to alter the surface chemistry. Surface chemical properties that can be modified with laser techniques include (but are not limited to) functional groups, ionization, moieties, and/or other chemical makeup. Laser techniques can be used to apply coating layers and/or grafting to at least one component of cardiac repair devices. Laser assisted chemical groups grafting may change hydrophilicity and/or hydrophobicity of the surface and/or electrostatic (cationic or ionic) nature of the substrate. In some instances, bovine pericardial tissues can be modified using laser grafting of hydrophilic polymers including (but not limited to) hyaluronic acid (HA), pure collagen, polysulfone, polyacrylate-based zwitterionic coatings polypeptides, polysaccharides, collagens, chitosan, silk, wool, cellulose, starches, pectin, gelatin, alginates, polyvinyl alcohol, polyethylene glycol ether, polyamides and urethane-based polymers.

In some instances, antimicrobial ability can be added by creating cationic groups on the surface of the substrate. Certain cationic chemical groups can bind and interact with the negatively charged bacterial cell membranes, leading to the change of the electrochemical potential on bacterial cell membranes, inducing cell membrane damage and the permeation of larger molecules such as proteins, destroying cell morphology and membranes and eventually resulting in cell death. Specific examples of the polymer coatings that can be activated or modified with lasers include (but are not limited to) methacrylates, polyurethanes, polyimides with phosphorylcholine, cysteine, sulfobetaine, carboxybetaine functional groups. Laser processes may form crosslinked polyzwitterionic acids.

Various laser sources can be used for surface chemistry modification processes in accordance with many examples. The laser sources with various emission wavelengths including (but not limited to) ultraviolet (UV), visible (vis), near-infrared (NIR), and infrared (IR) lasers, can be used for the modification. Examples of UV lasers include the ones based on the formation of excimer species in the gas phase. Examples of excimer lasers include the ArF, and the KrF, with emission wavelengths centered around 193 nm and around 248 nm, respectively. Laser materials modification techniques include (but are not limited to) quenching, annealing, re-melting, alloying, cladding, shock peening, glazing, texturing, and ablation.

While various processes for laser assisted surface coating are described above, any variety of processes that utilize laser techniques to apply surface coatings can be utilized in the biomedical devices as appropriate to the requirements of specific applications in accordance with various examples of the invention. For the purposes of illustrates a specific example of laser modifications on a prosthetic heart valve in accordance with various examples of the invention are discussed further below.

### Laser Modification of Prosthetic Valves

Many instances provide laser modification including (but not limited to) patterning of various patterns and/or shapes, laser ablation, laser assisted crosslink, and laser assisted surface coating, can be applied to at least one surface of cardiac repair and regeneration devices. Laser patterning modification with micropatterns and nanopatterns in accordance with some instances can increase biocompatibility of various components of prosthetic valves including (but not limited to) leaflets, stents, and skirts. Laser ablation can create patterns and/or create various thickness (contouring) across the biopolymers and/or the biological materials used in prosthetic valves. Laser assisted crosslink and/or grafting can enable localized and/or gradient crosslink or specific chemistry of the biological materials used in the prosthetic valves to reinforce the mechanical properties. The modified devices and/or the modified device surfaces may promote tissue ingrowth and endothelization. Examples of the devices include (but are not limited to) prosthetic heart valves, prosthetic stents, cardiac patches, vascular patches, annuloplasty devices and/or rings, stents, and/or any other system, where textured surfaces can increase successful implantation, repair, and/or remodeling.

Prosthetic valves in accordance with many instances can include many components constructed of various materials. For example, the frame can be rigid (e.g., constructed of a metal or metal alloy) for a valve to hold its shape and form the body of the prosthetic heart valve. Additional features include an inner material forming the functional aspects of a valve, such as leaflets. Leaflets can be made of biomaterials including (but not limited to) bovine pericardium tissue. Further examples include a skirt, which can extend on the inside or outside of the valve, which can provide a softer interface against native tissue on the downstream edge of the prosthetic heart valve. Skirts can be made of synthetic materials including (but not limited to) polymers and polyethylene terephthalate. The various materials (e.g., frame, inner material, and skirt) are typically selected for the properties selected above, which makes the use of a single material unlikely or impossible in the construction of prosthetic heart valve.

A significant obstacle in prosthetic valves and stents can be tissue rejection and/or damage caused by the implantation of a prosthesis. One method to improve acceptance is to promote tissue ingrowth into certain components of the prosthesis. In some instances, certain components of a prosthetic valve including (but not limited to) the skirt can be modified with laser techniques to promote the formation of fibrotic tissue, which can help maintain a position of the prosthesis. Some examples show that some components including (but not limited to) inner skirts may can be modified with laser techniques to promote endothelization. Endothelization can be beneficial for anti-hemolytic, anti-thrombotic, and anti-inflammatory activity against a prosthesis. Several examples provide that components including (but not limited to) frame may be modified with laser techniques to discourage or inhibit fibrosis and/or tissue overgrowth and/or provide a lubricous property to increase or improve manipulation, movement, and/or navigation. In certain examples, the patterning (e.g., micropatterning and/or nanopatterning) can be the same or different on the inner skirt and outer skirt.

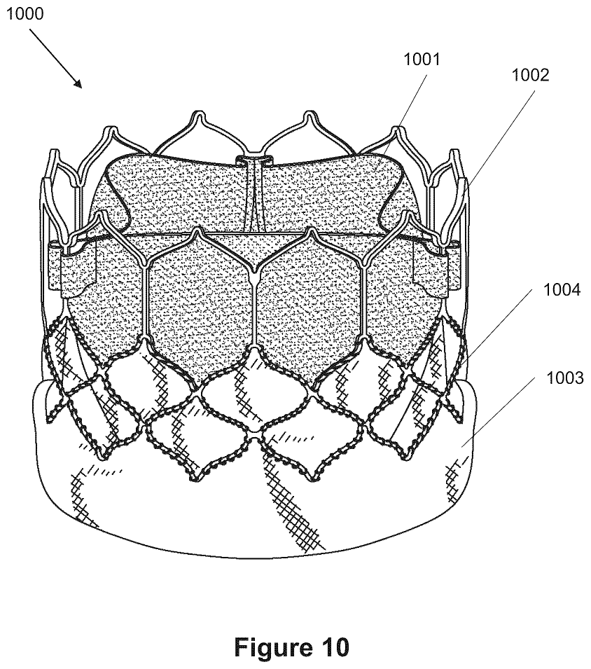

Some examples show that the components of the bioprosthetic transcatheter valve can be modified using various laser techniques. FIG. 10 illustrates laser modifications of a prosthetic transcatheter valve in accordance with an example. The prosthetic transcatheter valve 1000 can include the leaflets 1001, the expandable stent 1002, the skirts 1003, and the sutures 1004. The leaflets 1001 can be made of bovine and/or porcine pericardium tissues. Laser ablation in accordance with many examples can be applied to leaflets to achieve consistent thickness throughout the leaflets. Laser crosslinking with riboflavin in accordance with several examples can be applied to the leaflets to improve mechanical properties. The expandable stents 1002 can be made of nitinol or titanium. Laser micromachining of the nitinol stent in accordance with some examples can create nano- and micro-grooves on the outside surfaces of the stent. The modifications on the stent may allow for an easier tissue ingrowth to prevent PVL leaks. The skirts 1003 can be made of polyester or polyethylene terephthalate (PET). Laser ablation in accordance with certain examples can cause local melting on the surface of the skirt materials. The melting may reduce the tendency of the individual threads of the polyester or PET fabrics from becoming loose and protruding within the product. In addition, laser beam radiation can be applied to the skirt to modify the surface energy with the use of photoreagents. Surface modification of the skirts may decrease the contact angle of the materials thus improve the hydrophilicity. The sutures 1004 can be used to secure the skirts onto the expandable stents. The sutures can be made of PTFE and PET. Laser beam radiation can be applied to the sutures to modify the surface energy, with the use of photoreagents. Surface modification of the sutures may decrease the contact angle of the materials thus improve the hydrophilicity.

Additional examples are directed to cardiac repair devices and techniques, including (but not limited to) cardiac patches, vascular patches, annuloplasty devices and/or rings, stents, and/or any other system, where textured surfaces can increase successful implantation, repair, and/or remodeling.

### Data and Testing

The following section provides specific examples of the use of different laser modification processes to modify various components of bioprosthetic devices. It will be understood that the specific examples are provided for exemplary purposes and are not limiting to the overall scope of the disclosure, which must be considered in light of the entire specification, figures and claims.

Laser Assisted Crosslink with Riboflavin

Bovine pericardium tissues can be crosslinked with riboflavin using lasers. Laser crosslink processes can eliminate the use of toxic glutaraldehyde during crosslinking processes. The laser lights can enable localized crosslinking sites.

Before the laser treatment, bovine pericardium tissue can be soaked in a solution comprising riboflavin with cyclodextrin dissolved in saline or ethylene diamine tetraacetic acid (EDTA). Cyclodextrin to riboflavin can be about 25:1 ratio. Cyclodextrin can range between about 2% and about 3% by total weight; EDTA can be of about 0.05% by total weight; and salt can be about 0.25% by total weight. pH value of the solution may range between about 7.0 and about 7.5. Bovine pericardial tissue can be soaked in the riboflavin cylodextrin solution for about 2 hours at about 37° C. prior to laser crosslinking.

### Laser Surface Modification

Laser beam radiation can be applied to skirts and/or sutures of bioprosthetic valves to change the surface chemical composition and/or change the hydrophilicity. Surface energy modification can be carried out with Nd:YAG pulse laser (wavelength=266 nm, pulse width=ns) with triethylamine photo-reagent. Aqueous triethylamine (TEA) solution (TEA wt % about 6.5-6.7 in H₂O) can be applied to the polyester skirt of the surgical valve and/or the PTFE sutures. Laser beam radiation can then be applied to modify surface energy of the materials. Surface energy change can be confirmed by contact angle measurements. PTFE and PET both show a decrease in the contact angle and a change in the surface chemical composition after the irradiation.

### Doctrine of Equivalents

For purposes of this description, certain aspects, advantages, and novel features of the instances of this disclosure are described herein. The disclosed methods, apparatus, and systems should not be construed as being limiting in any way. Instead, the present disclosure is directed toward all novel and nonobvious features and aspects of the various disclosed instances, alone and in various combinations and sub-combinations with one another. The methods, apparatus, and systems are not limited to any specific aspect or feature or combination thereof, nor do the disclosed instances require that any one or more specific advantages be present or problems be solved.

Although the operations of some of the disclosed instances are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Reference to an object in the singular is not intended to mean “one and only one” unless explicitly so stated, but rather “one or more.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to +10% of that numerical value, such as less than or equal to +5%, less than or equal to +4%, less than or equal to +3%, less than or equal to +2%, less than or equal to +1%, less than or equal to +0.5%, less than or equal to +0.1%, or less than or equal to +0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In view of the many possible instances to which the principles of the disclosed technology may be applied, it should be recognized that the illustrated instances are only preferred examples and should not be taken as limiting the scope of the disclosure. Rather, the scope of the disclosure is at least as broad as the following claims.

### EXAMPLES

Example 1: An example of a method to improve biocompatibility of a structure for use in a prosthetic comprising:
applying a laser process to at least one surface of the structure to create at least one pattern on the at least one surface;
wherein the laser process comprises at least one laser source;
wherein the laser process is selected from the group consisting of direct laser writing, interference lithography, and any combinations thereof; and
wherein the structure is at least a component of a device selected from the group consisting of a prosthetic heart valve, a stent, and a cardiac patch.

Example 2: The example method of example 1, wherein the component is selected from the group consisting of a frame, a stent, a skirt, an outer skirt, an inner skirt, a suture, a leaflet, and a valve tissue.

Example 3: The example method of example 1 or 2, wherein the surface comprises a material selected from the group consisting of a metal, a metal alloy, a stainless steel, nitinol, titanium, Co—Cr alloy, a polymer, polymethylmethacrylate, polyetherketone, polyimide, polyamide, polyethylene, polytetrafluorethylene, nylon, polydimethylsiloxane, silicone, polyethylene terephthalate, polybutylene terephthalate, polyester, biopolymer, a blocked copolymer of polycarbonate, a poly(sulfone of bisphenol-A) (PSU)-PBT copolymer, collagen, acrylate collagen, chitosan, and a pericardial tissue.

Example 4: The example method of example 1, 2, or 3, wherein the at least one laser source is an ultrashort pulse laser.

Example 5: The example method of any one of examples 1 to 4, wherein the ultrashort pulse laser has a pulse width from 3 picoseconds to 50 femtoseconds.

Example 6: The example method of any one of examples 1 to 5, wherein the at least one laser source has an emission wavelength selected from the group consisting of an infrared wavelength from 700 nm to 1 mm, a near infrared wavelength from 800 nm to 2500 nm, a visible light wavelength from 380 nm to 750 nm, and an ultraviolet wavelength from 100 nm to 400 nm.

Example 7: The example method of any one of examples 1 to 6, wherein the at least one laser source has an emission wavelength selected from the group consisting of 10.6 μm, 1060 nm, 1030 nm, 530 nm, 515 nm, 370 nm, 355 nm, 343 nm, 248 nm, and 193 nm.

Example 8: The example method of any one of examples 1 to 7, wherein the direct laser writing is carried out using a direct laser writing system comprising at least one laser beam, at least one substrate, and at least one galvaometric mirror.

Example 9: The example method of example 8, wherein the at least one substrate is fixed and the at least one galvaometric mirror moves the at least one laser beam to create the plurality of patterns; or the at least one laser beam is fixed and the at least one substrate moves to create a plurality of patterns; or the at least one substrate and the at least one laser beam move simultaneously to create the plurality of patterns.

Example 10: The example method of example 8 or 9, wherein the direct laser writing system comprises a focusing optic selected from the group consisting of a microscope objective, and an f-theta lens.

Example 11: The example method of any one of examples 1 to 10, wherein the at least one pattern improves reendothelization and tissue regeneration of the structure.

Example 12: The example method of any one of examples 1 to 11, wherein the laser process changes at least one property selected from the group consisting of surface topography, thickness, and dimension, of the structure.

Example 13: The example method of any one of examples 1 to 12, wherein the at least one surface is flat, curved, even, or uneven.

Example 14: The example method of any one of examples 1 to 13, wherein the at least one pattern is periodic or aperiodic.

Example 15: The example method of any one of examples 1 to 14, wherein the at least one pattern has at least one dimension in a range from 1 nm to 1 mm.

Example 16: The example method of any one of examples 1 to 15, wherein the laser process is a part of a subtractive process or an additive process.

Example 17: The example method of any one of examples 1 to 16, wherein the laser process is a laser ablation process, wherein the at least one pattern changes a thickness of the at least one surface.

Example 18: The example method of example 17, wherein the laser ablation process contours and creates different thickness on the at least one surface.

Example 19: The example method of any one of examples 1 to 18, wherein the at least one pattern comprises a hierarchical structure or is multi-dimensional.

Example 20: The example method of any one of examples 1 to 19, wherein the at least one pattern comprises a pattern selected from the group consisting of a line, a straight line, a curved line, a groove, a pillar, a pore, a ridge, a wave, a dimple, a square, and any combinations thereof.

Example 21: The example method of any one of examples 1 to 20, wherein the at least one pattern has at least one shape selected from the group consisting of circular, ovular, oblong, triangular, quadrilateral, rectangular, square, rhomboidal, trapezoidal, hexagonal, octagonal, and any combinations thereof.

Example 22: The example method of any one of examples 1 to 21, wherein the at least one pattern comprises parallel rows.

Example 23: The example method of any one of examples 1 to 22, further comprising applying at least one chemical reagent to the at least one surface before applying the laser process, wherein the laser process generates a surface coating of the at least one surface.

Example 24: The example method of example 23, wherein the surface coating changes the contact angle of the at least one surface.

Example 25: An example of a method to modify a biomaterial for use within a prosthetic comprising:
adding a photoactive reagent to a proteinaceous material; and
applying a laser process comprising at least one laser source onto the proteinaceous material;
wherein the at least one laser source locally crosslinks the photoactive reagent with the proteinaceous material and changes a chemical structure of the proteinaceous material; and
wherein the proteinaceous material is at least a component of a system or device selected from the group consisting of a prosthetic heart valve, a stent, and a cardiac patch.

Example 26: The example method of example 25, wherein the photoactive reagent is selected from the group consisting of aryl azide, azido-methyl-coumarin, benzophenone, anthraquinone, diazo compound, diazirines psoralen derivative, vitamin B1 (thiamine), vitamin B2 (riboflavin), vitamin B3 (niacin), vitamin B5 (pantothenic acid), vitamin B6 (pyridoxine), vitamin B7 (biotin), vitamin B12 (cobalamin), folic acid, n-hydroxy succinimide ester of acrylic acid (ANHS), 2-hydroxy-4′-(2-hydroxyethoxy)-2-methylpropiophenone (Irgacure 2959 photoinitiator), methyl phenylglyoxylate, phenylbis (2,4,6-trimethylbenzoyl) phosphine oxide, sulfo-LC-SDA (Diazirine), triethylamine, deoxyribonucleic acid, bis-acylphosphine oxide, pyrolipid, porphyrin, and chlorin.

Example 27: The example method of example 25 or 26, the photoactive reagent is added to the proteinaceous material by soaking the proteinaceous material in a solution comprising the photoactive reagent; or by surface coating the proteinaceous material with a solution comprising the photoactive reagent.

Example 28: The example method of example 25, 26, or 27, wherein the photoactive reagent is riboflavin and the proteinaceous material comprises collagen.

Example 29: The example method of any one of examples 25 to 28, wherein the at least one laser source is an ultrashort pulse laser.

Example 30: The example method of any one of examples 25 to 29, wherein the at least one laser source has an emission wavelength from 100 nm to 400 nm.

Example 31: The example method of any one of examples 25 to 30, wherein the crosslink changes at least one mechanical property of the biomaterial selected from the group consisting of ultimate tensile strength, fatigue strength, and Young's modulus.

Example 32: The example method of any one of examples 25 to 31, wherein the laser process creates a gradient of crosslinking in the proteinaceous material by controlling the at least one laser source.

Example 33: An example of a prosthetic heart valve, comprising:
an annular frame that is radially collapsible to a collapsed configuration and radially expandable to an expanded configuration, the frame having an inflow end and an outflow end, and defining a longitudinal axis along a lumen of the prosthetic heart valve when the prosthetic heart valve is in the expanded configuration;
a leaflet structure positioned within the frame and secured thereto; and a skirt comprising an inner skirt positioned on the inside of the frame and an outer skirt positioned on the outside of the frame, the inner skirt and the outer skirt being attached to at least a portion of the frame by a plurality of sutures;
wherein, on the outside of the frame, the outer skirt extends along the longitudinal axis in an upstream direction and doubles back toward the outflow end of the frame at a fold line to form a cuff, and an edge portion of the outer skirt is secured to the outer skirt downstream of the fold line such that the cuff forms an inflow end of the laminate sealing member;
wherein at least one surface of: the frame, the leaflet, the skirt, and the suture, is modified by a laser process comprising at least one laser source; and
wherein the laser process improves reendothelization and tissue regeneration of the prosthetic heart valve.

Example 34: The example method of example 33, wherein the laser process is selected from the group consisting of direct laser writing, interference lithography, and any combinations thereof.

Example 35: The example method of example 33 or 34, wherein the at least one laser source is an ultrashort pulse laser.

Example 36: The example method of example 35, wherein the ultrashort pulse laser has a pulse width from 1 millisecond to 1 femtosecond.

Example 37: The example method of any one of examples 33 to 36, wherein the at least one laser source has an emission wavelength selected from the group consisting of an infrared wavelength from 700 nm to 1 mm, a near infrared wavelength from 800 nm to 2500 nm, a visible light wavelength from 380 nm to 750 nm, and an ultraviolet wavelength from 100 nm to 400 nm.

Example 38: The example method of any one of examples 33 to 37, wherein the at least one laser source has an emission wavelength selected from the group consisting of 10.6 μm, 1060 nm, 1030 nm, 530 nm, 515 nm, 370 nm, 355 nm, 343 nm, 248 nm, and 193 nm.

Example 39: The example method of any one of examples 33 to 38, wherein the direct laser writing is carried out using a direct laser writing system comprising at least one laser beam, at least one substrate, and at least one galvaometric mirror.

Example 40: The example method of any one of examples 33 to 39, wherein the at least one substrate is fixed and the at least one galvaometric mirror moves the at least one laser beam to create a plurality of patterns; or the at least one laser beam is fixed and the at least one substrate moves to create the plurality of patterns; or the at least one substrate and the at least one laser beam move simultaneously to create the plurality of patterns.

Example 41: The example method of any one of examples 33 to 40, wherein the direct laser writing system comprises a focusing optic selected from the group consisting of a microscope objective, and an f-theta lens.

Example 42: The example method of any one of examples 33 to 41, wherein the at least one surface is flat, curved, even, or uneven.

Example 43: The example method of any one of examples 33 to 42, wherein the at least one surface comprises at least one pattern created by the laser process.

Example 44: The example method of example 43, wherein the at least one pattern is periodic or aperiodic.

Example 45: The example method of example 43 or 44, wherein the at least one pattern has at least one dimension in a range from 1 nm to 1 mm.

Example 46: The example method of example 43, 44, or 45, wherein the at least one pattern comprises a hierarchical structure or is multi-dimensional.

Example 47: The example method of any one of examples 43 to 46, wherein the at least one pattern is selected from the group consisting of a line, a straight line, a curved line, a groove, a pillar, a pore, a ridge, a wave, a dimple, a square, and any combinations thereof.

Example 48: The example method of any one of examples 43 to 47, wherein the at least one pattern has at least one shape selected from the group consisting of circular, ovular, oblong, triangular, quadrilateral, rectangular, square, rhomboidal, trapezoidal, hexagonal, octagonal, and any combinations thereof.

Example 49: The example method of any one of examples 43 to 48, wherein the at least one pattern comprises parallel rows.

Example 50: The example method of any one of examples 33 to 49, wherein the laser process is a part of a subtractive process or an additive process.

Example 51: The example method of any one of examples 33 to 50, wherein the laser process is a laser ablation process, wherein the laser ablation process changes a thickness of the at least one surface.

Example 52: The example method of example 51, wherein the laser ablation process contours and creates different thickness on the at least one surface.

Example 53: The example method of any one of examples 33 to 52, wherein the laser process generates a surface coating on the at least one surface with at least one chemical reagent applied to the at least one surface.

Example 54: The example method of example 53, wherein the surface coating changes a contact angle of the at least one surface.

Example 55: The example method of any one of examples 33 to 54, wherein the leaflet comprises a pericardial tissue and the laser process generates a consistent thickness throughout the leaflet.

Example 56: The example method of any one of examples 33 to 55, wherein the laser process creates a pattern on the at least one surface of the frame and the pattern allows an easy tissue ingrowth and prevents paravalvular leak.

Example 57: The example method of any one of examples 33 to 56, wherein the laser process modifies a surface energy and changes a contact angle of the at least one surface of the skirt or the suture.

Example 58: An example of a prosthetic heart valve, comprising:
an annular frame that is radially collapsible to a collapsed configuration and radially expandable to an expanded configuration, the frame having an inflow end and an outflow end, and defining a longitudinal axis along a lumen of the prosthetic heart valve when the prosthetic heart valve is in the expanded configuration;
a leaflet structure positioned within the frame and secured thereto; and
a skirt comprising an inner skirt positioned on the inside of the frame and an outer skirt positioned on the outside of the frame, the inner skirt and the outer skirt being attached to at least a portion of the frame by a plurality of sutures;
wherein, on the outside of the frame, the outer skirt extends along the longitudinal axis in an upstream direction and doubles back toward the outflow end of the frame at a fold line to form a cuff, and an edge portion of the outer skirt is secured to the outer skirt downstream of the fold line such that the cuff forms an inflow end of the laminate sealing member;
wherein a laser process comprising at least one laser source locally crosslinks the leaflet with a photoactive reagent; and
wherein the leaflet comprises a proteinaceous material, and the laser process changes a chemical structure of the proteinaceous material.

Example 59: The example method of example 58, wherein the photoactive reagent is selected from the group consisting of aryl azide, azido-methyl-coumarin, benzophenone, anthraquinone, diazo compound, diazirines psoralen derivative, vitamin B1 (thiamine), vitamin B2 (riboflavin), vitamin B3 (niacin), vitamin B5 (pantothenic acid), vitamin B6 (pyridoxine), vitamin B7 (biotin), vitamin B12 (cobalamin), folic acid, n-hydroxy succinimide ester of acrylic acid (ANHS), 2-hydroxy-4′-(2-hydroxyethoxy)-2-methylpropiophenone (Irgacure 2959 photoinitiator), methyl phenylglyoxylate, phenylbis (2,4,6-trimethylbenzoyl) phosphine oxide, sulfo-LC-SDA (Diazirine), triethylamine, deoxyribonucleic acid, bis-acylphosphine oxide, pyrolipid, porphyrin, and chlorin.

Example 60: The example method of example 58 or 59, the photoactive reagent is added to the proteinaceous material by soaking the proteinaceous material in a solution comprising the photoactive reagent; or by surface coating the proteinaceous material with a solution comprising the photoactive reagent.

Example 61: The example method of example 58, 59, or 60, wherein the photoactive reagent is riboflavin and the proteinaceous material comprises collagen.

Example 62: The example method of any one of examples 58 to 61, wherein the at least one laser source is an ultrashort pulse laser.

Example 63: The example method of any one of examples 58 to 62, wherein the at least one laser source has an emission wavelength from 100 nm to 400 nm.

Example 64: The example method of any one of examples 58 to 63, wherein the crosslink changes at least one mechanical property of the material selected from the group consisting of ultimate tensile strength, fatigue strength, and Young's modulus.

Example 65: The example method of any one of examples 58 to 64, wherein the laser process creates a gradient of crosslinking in the proteinaceous material by controlling the at least one laser source.

What is claimed is:

1. A method to improve biocompatibility of a structure for use in a prosthetic comprising:
applying a laser process to at least one surface of the structure to create at least one pattern on the at least one surface;
wherein the laser process comprises at least one laser source;
wherein the laser process is selected from the group consisting of direct laser writing, interference lithography, and any combinations thereof; and
wherein the structure is at least a component of a device selected from the group consisting of a prosthetic heart valve, a stent, and a cardiac patch.

2. The method of claim 1, wherein the surface comprises a material selected from the group consisting of a metal, a metal alloy, a stainless steel, nitinol, titanium, Co—Cr alloy, a polymer, polymethylmethacrylate, polyetherketone, polyimide, polyamide, polyethylene, polytetrafluorethylene, nylon, polydimethylsiloxane, silicone, polyethylene terephthalate, polybutylene terephthalate, polyester, biopolymer, a blocked copolymer of polycarbonate, a poly(sulfone of bisphenol-A) (PSU)-PBT copolymer, collagen, acrylate collagen, chitosan, and a pericardial tissue.

3. The method of claim 1, wherein the at least one laser source is an ultrashort pulse laser.

4. The method of claim 3, wherein the ultrashort pulse laser has a pulse width from 3 picoseconds to 50 femtoseconds.

5. The method of claim 1, wherein the at least one laser source has an emission wavelength selected from the group consisting of an infrared wavelength from 700 nm to 1 mm, a near infrared wavelength from 800 nm to 2500 nm, a visible light wavelength from 380 nm to 750 nm, and an ultraviolet wavelength from 100 nm to 400 nm.

6. The method of claim 1, wherein the direct laser writing is carried out using a direct laser writing system comprising at least one laser beam, at least one substrate, and at least one galvaometric mirror; wherein the at least one substrate is fixed and the at least one galvaometric mirror moves the at least one laser beam to create the plurality of patterns; or the at least one laser beam is fixed and the at least one substrate moves to create a plurality of patterns; or the at least one substrate and the at least one laser beam move simultaneously to create the plurality of patterns; and wherein the direct laser writing system comprises a focusing optic selected from the group consisting of a microscope objective, and an f-theta lens.

7. The method of claim 1, wherein the at least one pattern improves reendothelization and tissue regeneration of the structure.

8. The method of claim 1, wherein the laser process changes at least one property selected from the group consisting of surface topography, thickness, and dimension, of the structure.

9. The method of claim 1, wherein the laser process is a laser ablation process, wherein the at least one pattern changes a thickness of the at least one surface; and wherein the laser ablation process contours and creates different thickness on the at least one surface.

10. The method of claim 1, further comprising applying at least one chemical reagent to the at least one surface before applying the laser process, wherein the laser process generates a surface coating of the at least one surface; and wherein the surface coating changes the contact angle of the at least one surface.

11. A prosthetic heart valve, comprising:
an annular frame that is radially collapsible to a collapsed configuration and radially expandable to an expanded configuration, the frame having an inflow end and an outflow end, and defining a longitudinal axis along a lumen of the prosthetic heart valve when the prosthetic heart valve is in the expanded configuration;
a leaflet structure positioned within the frame and secured thereto; and
a skirt comprising an inner skirt positioned on the inside of the frame and an outer skirt positioned on the outside of the frame, the inner skirt and the outer skirt being attached to at least a portion of the frame by a plurality of sutures;
wherein, on the outside of the frame, the outer skirt extends along the longitudinal axis in an upstream direction and doubles back toward the outflow end of the frame at a fold line to form a cuff, and an edge portion of the outer skirt is secured to the outer skirt downstream of the fold line;
wherein at least one surface of: the frame, the leaflet, the skirt, and the suture, is modified by a laser process comprising at least one laser source; and
wherein the laser process improves reendothelization and tissue regeneration of the prosthetic heart valve.

12. The prosthetic heart valve of claim 11, wherein the laser process is selected from the group consisting of direct laser writing, interference lithography, and any combinations thereof.

13. The prosthetic heart valve of claim 11, wherein the at least one laser source is an ultrashort pulse laser.

14. The prosthetic heart valve of claim 13, wherein the ultrashort pulse laser has a pulse width from 1 millisecond to 1 femtosecond.

15. The prosthetic heart valve of claim 11, wherein the at least one laser source has an emission wavelength selected from the group consisting of an infrared wavelength from 700 nm to 1 mm, a near infrared wavelength from 800 nm to 2500 nm, a visible light wavelength from 380 nm to 750 nm, and an ultraviolet wavelength from 100 nm to 400 nm.

16. The prosthetic heart valve of claim 11, wherein the direct laser writing is carried out using a direct laser writing system comprising at least one laser beam, at least one substrate, and at least one galvaometric mirror; wherein the at least one substrate is fixed and the at least one galvaometric mirror moves the at least one laser beam to create a plurality of patterns; or the at least one laser beam is fixed and the at least one substrate moves to create the plurality of patterns; or the at least one substrate and the at least one laser beam move simultaneously to create the plurality of patterns; and wherein the direct laser writing system comprises a focusing optic selected from the group consisting of a microscope objective, and an f-theta lens.

17. The prosthetic heart valve of claim 11, wherein the laser process is a laser ablation process, wherein the laser ablation process changes a thickness of the at least one surface; and wherein the laser ablation process contours and creates different thickness on the at least one surface.

18. The prosthetic heart valve of claim 11, wherein the laser process generates a surface coating on the at least one surface with at least one chemical reagent applied to the at least one surface; and wherein the surface coating changes a contact angle of the at least one surface.

19. The prosthetic heart valve of claim 11, wherein the leaflet comprises a pericardial tissue and the laser process generates a consistent thickness throughout the leaflet.

20. The prosthetic heart valve of claim 11, wherein the laser process creates a pattern on the at least one surface of the frame and the pattern allows an easy tissue ingrowth and prevents paravalvular leak.
